(19) United States
(12) Patent Application Publication
(10) Pub. No.: US 2023/0203191 A1
(43) Pub. Date: Jun. 29, 2023

(54) ENGINEERED ANTIBODIES

(71) Applicant: DANISCO US INC, Palo Alto, CA (US)

(72) Inventors: David A. ESTELL, Palo Alto, CA (US); Amy Deming LIU, Sunnyvale, CA (US); Michael C. MILLER, Palo Alto, CA (US); Jeffrey Wayne MUNOS, Palo Alto, CA (US); Jonathan David PETERSON, Egg Harbor Twp, NJ (US)

(21) Appl. No.: 17/915,883

(22) PCT Filed: Mar. 30, 2021
(86) PCT No.: PCT/US2021/024807

§ 371 Date: Sep. 29, 2022

Related U.S. Application Data
(60) Provisional application No. 63/001,736, filed on Mar. 30, 2020.

(51) Int. Cl. C07K 16/32; C07K 16/10

(52) U.S. Cl. CPC C07K 16/32; C07K 16/1063; C07K 2317/53; C07K 2317/54; C07K 2317/55; C07K 2317/94; C07K 2317/24

(57) ABSTRACT

Provided herein, inter alia, are engineered antibodies or antigen-binding fragments thereof that exhibit one or more improved properties relating to manufacturability, thermostability, and/or protease resistance as well as methods for making and using the same.

### CROSS-REFERENCE TO RELATED APPLICATIONS

This application claims priority to U.S. Provisional Patent Application No. 63/001,736, filed Mar. 30, 2020, the disclosure of which is incorporated by reference herein in its entirety.

### FIELD OF THE INVENTION

Provided herein, inter alia, are engineered antibodies and antigen-binding fragments thereof having one or more improved properties such as increased manufacturability, thermostability, and/or protease resistance as well as methods for making and using the same.

### BACKGROUND

Antibody-based therapeutics have been used successfully to treat a variety of diseases, including cancer and autoimmune/inflammatory disorders. Antibodies also play a role in diagnostics and other medical devices. Large-scale manufacturing of antibodies is hindered due to limits in the amount of host cell protein production (primarily human embryonic kidney cells (HEK) and Chinese Hamster Ovary (CHO) cells), poor thermostability of manufactured antibodies, and susceptibility to proteolytic cleavage. There is thus a need for improved alternative technologies for producing antibody-based biological therapeutics which result in improvements in manufacturability. Ideally, these improved technologies would result in a more stable, better folded, and better antigen-binding antibody capable of large-scale production at cost and volume targets that will enable their use as traditional therapeutics and diagnostics but also for use as topical and oral products for human and animal health.

The subject matter disclosed herein addresses these needs and provides additional benefits as well.

### SUMMARY

Provided herein, inter alia, are engineered antibodies containing one or more amino acid substitution(s) that result in one or more improved properties compared to an identical antibody that does not comprise one or more amino acid substitution(s) as well as methods for making and using the same.

Accordingly, in some aspects, provided herein is an isolated monoclonal antibody or functional fragment thereof comprising: (a) a heavy chain variable region comprising one or more amino acid substitution(s) comprising T or an I at Kabat position 5; N at Kabat position 50, V or Y at Kabat position 84; and/or S at Kabat position 85; and or (b) a heavy chain constant region comprising one or more amino acid substitution(s) comprising Q at position 17; V at position 18; V at position 64; L at position 151; C, E, or D at position 152; N at position 156; and/or P at position 157, wherein the amino acid positions of the heavy chain constant region correspond to those of SEQ ID NO:1, and wherein said antibody exhibits one or more improved properties comprising increased manufacturability, thermostability, and/or protease resistance compared to an antibody that does not comprise the one or more amino acid substitutions. In some embodiments, the antibody or functional fragment thereof further comprises: (c) a light chain variable region comprising an amino acid substitution comprising F at Kabat position 42; and/or (d) a light chain constant region comprising one or more amino acid substitution(s) comprising T at position 81; M or V at position 97; and/or I at position 100, wherein the amino acid positions of the light chain constant region correspond to those of SEQ ID NO:2. In other aspects, provided herein is an isolated monoclonal antibody or functional fragment thereof comprising: (c) a light chain variable region comprising an amino acid substitution comprising F at Kabat position 42; and/or (d) a light chain constant region comprising one or more amino acid substitution(s) comprising T at position 81; M or V at position 97; and/or I at position 100, wherein the amino acid positions of the light chain constant region correspond to those of SEQ ID NO:2, wherein said antibody exhibits one or more improved properties comprising increased manufacturability, thermostability, and/or protease resistance compared to an antibody that does not comprise the one or more amino acid substitutions. In some embodiments, the antibody or functional fragment thereof further comprises: (a) a heavy chain variable region comprising one or more amino acid substitution(s) comprising T or an I at Kabat position 5; N at Kabat position 50, V or Y at Kabat position 84; and/or S at Kabat position 85; and or (b) a heavy chain constant region comprising one or more amino acid substitution(s) comprising Q at position 17; V at position 18; V at position 64; L at position 151; C, E, or D at position 152; N at position 156; and/or P at position 157, wherein the amino acid positions of the heavy chain constant region correspond to those of SEQ ID NO:1.

In some embodiments of any of the embodiments disclosed herein, the antibody or functional fragment thereof of any one of claims 1-4, wherein said functional fragment is selected from the group consisting of Fab, Fab′, F(ab′)2 and Fv fragments. In some embodiments of any of the embodiments disclosed herein, said antibody is chimeric, humanized, or fully human. In some embodiments of any of the embodiments disclosed herein, said antibody is selected from the group consisting of trastuzumab, an anti-HSV8 antibody, and VRC01. In some embodiments of any of the embodiments disclosed herein, said antibody competitively inhibits the binding of one or more of trastuzumab, an anti-HSV8 antibody, and VRCO1 to an antigen.

In other aspects, provided herein is a nucleic acid encoding any of the heavy chain variable region and/or heavy chain constant region disclosed herein. In other aspects, provided herein is a nucleic acid encoding any of the light chain variable region and/or light chain constant region of disclosed herein.

In further aspects, provided herein is a vector comprising any of the nucleic acids disclosed herein.

In yet additional aspects, provided herein is a recombinant cell comprising any of the vectors disclosed herein. In some embodiments, the cell is a mammalian cell, a bacterial cell, or a fungal cell. In some embodiments, the fungal cell is T. reesei.

In still other aspects, provided herein is a method for improving one or more properties in a monoclonal antibody comprising: (a) introducing one or more substitutions comprising T or an I at Kabat position 5; N at Kabat position 50, V or Y at Kabat position 84; and/or S at Kabat position 85 in a heavy chain variable region; and/or (b) introducing one or more substitutions comprising Q at position 17; V at position 18; V at position 64; L at position 151; C, E, or D at position 152; N at position 156; and/or P at position 157, in a heavy chain constant region wherein the amino acid positions of the heavy chain constant region correspond to those of SEQ ID NO:1. In some embodiments, the method further comprises: (c) introducing one or more substitutions comprising F at Kabat position 42 in a light chain variable region; and/or (d) introducing one or more substitutions T at position 81; M or V at position 97; and/or I at position 100 in a light chain constant region, wherein the amino acid positions of the light chain constant region correspond to those of SEQ ID NO:2. In another aspect, provided herein is a method for improving one or more properties in a monoclonal antibody comprising: (c) introducing a substitution comprising F at Kabat position 42 in a light chain variable region; and/or (d) introducing one or more substitution(s) comprising T at position 81; M or V at position 97; and/or I at position 100 in a light chain constant region, wherein the amino acid positions of the light chain constant region correspond to those of SEQ ID NO:2. In some embodiments, the method further comprises: (a) introducing one or more substitutions comprising T or an I at Kabat position 5; N at Kabat position 50, V or Y at Kabat position 84; and/or S at Kabat position 85 in a heavy chain variable region; and/or (b) introducing one or more substitutions comprising Q at position 17; V at position 18; V at position 64; L at position 151; C, E, or D at position 152; N at position 156; and/or P at position 157, in a heavy chain constant region wherein the amino acid positions of the heavy chain constant region correspond to those of SEQ ID NO:1. In some embodiments of any of the embodiments disclosed herein, said improved properties is one or more of increased manufacturability, thermostability, and/or protease resistance. In some embodiments of any of the embodiments disclosed herein, said antibody is selected from the group consisting of trastuzumab, an anti-HSV8 antibody, and VRC01.

In additional aspects, provided herein is a method for producing a monoclonal antibody or functional fragment thereof comprising providing an isolated cell with a nucleic acid encoding said antibody or functional part thereof, wherein said antibody or functional part thereof comprises one or more of: (a) a heavy chain variable region comprising one or more amino acid substitution(s) comprising T or an I at Kabat position 5; N at Kabat position 50, V or Y at Kabat position 84; and/or S at Kabat position 85; (b) a heavy chain constant region comprising one or more amino acid substitution(s) comprising Q at position 17; V at position 18; V at position 64; L at position 151; C, E, or D at position 152; N at position 156; and/or P at position 157, wherein the amino acid positions of the heavy chain constant region correspond to those of SEQ ID NO:1 (c) a light chain variable region comprising an amino acid substitution comprising F at Kabat position 42; and/or (d) a light chain constant region comprising one or more amino acid substitution(s) comprising T at position 81; M or V at position 97; and/or I at position 100, wherein the amino acid positions of the light chain constant region correspond to those of SEQ ID NO:2. In some embodiments, the cell is a mammalian cell, a bacterial cell, or a fungal cell. In some embodiments, the fungal cell is T. reesei. In some embodiments of any of the embodiments disclosed herein, the antibody or functional fragment thereof exhibits one or more improved properties selected from the group consisting of increased manufacturability, thermostability, and protease resistance compared to an antibody that does not comprise one or more of the amino acid substitutions. In some embodiments of any of the embodiments disclosed herein, said antibody is selected from the group consisting of trastuzumab, an anti-HSV8 antibody, and VRC01. In yet additional aspects, provided herein is a pharmaceutical composition comprising any of the antibodies or functional fragments thereof disclosed herein and a pharmaceutically acceptable carrier, diluent, or excipient.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### DETAILED DESCRIPTION

The overwhelming majority of antibody engineering has historically taken place in one or more of the antibody's complementarity-determining regions (CDRs), usually for purposes of improving antigen-epitope binding. CDRs are part of the variable chains in immunoglobulins and T cell receptors, generated by B-cells and T-cells respectively, where these molecules bind to their specific antigen. As the most variable parts of immunoglobulins, CDRs are crucial to the diversity of antigen specificities generated by lymphocytes.

Another region of immunoglobulins that has historically seen engineering is the Fc region. This region is composed of two heavy chains that contribute two or three constant domains depending on the class of the antibody. Thus, the Fc region ensures that each antibody generates an appropriate immune response for a given antigen, by binding to a specific class of Fc receptors, and other immune molecules, such as complement proteins. Amino acid engineering in this region, particularly for therapeutic antibodies, typically are for purposes of decreasing unwanted immunogenicity when administered to human patients.

As will be described in more detail herein, the inventors of the present application have surprisingly discovered a series of amino acid substitutions on the heavy chain and light chain of monoclonal antibodies that improve manufacturability, thermostability, and/or protease resistance. These substitutions are not located in the CDRs but, rather, in the framework regions separating the CDRs as well as in the Fc regions. Even more unexpectedly, these substitutions result in improved properties irrespective of the antibody backbone into which they are introduced. Thus, the amino acid substitutions disclosed herein do not decrease the stability or expression to levels below those of the parent antibody and can work in any antibody to result in a more stable, better folded, and more easily manufacturable immunoglobulin.

### I. Definitions

As used herein, “antibody” refers to immunoglobulins and immunoglobulin fragments, whether natural or partially or wholly synthetically, such as recombinantly, produced, including any fragment thereof containing at least a portion of the variable region of the immunoglobulin molecule that retains the binding specificity ability of the full-length immunoglobulin. Hence, an antibody includes any protein having a binding domain that is homologous or substantially homologous to an immunoglobulin antigen-binding domain (antibody combining site). Antibodies include antibody fragments. As used herein, the term antibody, thus, includes synthetic antibodies, recombinantly produced antibodies, multispecific antibodies (e.g., bispecific antibodies), human antibodies, non-human antibodies, humanized antibodies, chimeric antibodies, intrabodies, and antibody fragments, such as, but not limited to, Fab fragments, Fab′ fragments, F(ab′)2 fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the above. Antibodies provided herein include members of any immunoglobulin type (e.g., IgG, IgM, IgD, IgE, IgA and IgY), any class (e.g. IgG1, IgG2, IgG3, IgG4, IgAl and IgA2) or subclass (e.g., IgG2a and IgG2b).

As used herein, an “antibody fragment” or “antigen-binding fragment” of an antibody refers to any portion of a full-length antibody that is less than full length but contains at least a portion of the variable region of the antibody that binds antigen (e.g. one or more CDRs and/or one or more antibody combining sites) and thus retains the binding specificity, and at least a portion of the specific binding ability of the full-length antibody. Hence, an antigen-binding fragment refers to an antibody fragment that contains an antigen-binding portion that binds to the same antigen as the antibody from which the antibody fragment is derived. Antibody fragments include antibody derivatives produced by enzymatic treatment of full-length antibodies, as well as synthetically, e.g. recombinantly produced derivatives. An antibody fragment is included among antibodies. Examples of antibody fragments include, but are not limited to, Fab, Fab′, F(ab′)₂, single-chain Fv (scFv), Fv, dsFv, diabody, Fd and Fd′ fragments and other fragments, including modified fragments (see, for example, Methods in Molecular Biology, Vol 207: Recombinant Antibodies for Cancer Therapy Methods and Protocols (2003); Chapter 1; p 3-25, Kipriyanov). The fragment can include multiple chains linked together, such as by disulfide bridges and/or by peptide linkers. An antibody fragment generally contains at least or about 50 amino acids and typically at least or about 200 amino acids. An antigen-binding fragment includes any antibody fragment that when inserted into an antibody framework (such as by replacing a corresponding region) results in an antibody that immunospecifically binds (i.e. exhibits Ka of at least or at least about 10⁷-10⁸ M⁻¹) to the antigen.

As used herein, a “therapeutic antibody” refers to any antibody or antigen-binding fragment thereof that is administered for treatment of an animal, including a human. Such antibodies can be prepared by any known methods for the production of polypeptides, and hence, include, but are not limited to, recombinantly produced antibodies, synthetically produced antibodies, and therapeutic antibodies extracted from cells or tissues and other sources. As isolated from any sources or as produced, therapeutic antibodies can be heterogeneous in length or differ in post-translational modification, such as glycosylation (i.e. carbohydrate content). Heterogeneity of therapeutic antibodies also can differ depending on the source of the therapeutic antibodies. Hence, reference to therapeutic antibodies refers to the heterogeneous population as produced or isolated. When a homogeneous preparation is intended, it will be so-stated. References to therapeutic antibodies herein are to their monomeric, dimeric or other multimeric forms, as appropriate.

As used herein, a “neutralizing antibody” is any antibody or antigen-binding fragment thereof that binds to a pathogen and interferes with the ability of the pathogen to infect a cell and/or cause disease in a subject. Exemplary of neutralizing antibodies are neutralizing antibodies that bind to viruses, bacteria, and fungal pathogens. Typically, the neutralizing antibodies provide herein bind to the surface of the pathogen. In examples where the pathogen is a virus, a neutralizing antibody that binds to the virus typically binds to a protein on the surface of the virus. Depending on the class of the virus, the surface protein can be a capsid protein (e.g. a capsid protein of a non-enveloped virus) or a viral envelope protein (e.g., a viral envelope protein of an enveloped virus). In some examples, the protein is a glycoprotein. The ability of the virus to inhibit virus infectivity can be measure for example, by an in vitro neutralization assay, such as, for example, a plaque reduction assay using Vero host cells.

As used herein, a “conventional antibody” refers to an antibody that contains two heavy chains (which can be denoted H and H′) and two light chains (which can be denoted L and L′) and two antibody combining sites, where each heavy chain can be a full-length immunoglobulin heavy chain or any functional region thereof that retains antigen-binding capability (e.g. heavy chains include, but are not limited to, VH, chains VH-CH1 chains and VH-CH1-CH2-CH3 chains), and each light chain can be a full-length light chain or any functional region of (e.g. light chains include, but are not limited to, VL chains and VL-CL chains). Each heavy chain (H and H′) pairs with one light chain (L and L′, respectively)

As used herein, a full-length antibody is an antibody having two full-length heavy chains (e.g. VH-CH1-CH2-CH3 or VH-CH1-CH2-CH3-CH4) and two full-length light chains (VL-CL) and hinge regions, such as human antibodies produced naturally by antibody secreting B cells and antibodies with the same domains that are synthetically produced.

As used herein, the term “derivative” refers to a polypeptide that contains an amino acid sequence of an engineered antibody or a fragment thereof which has been modified, for example, by the introduction of amino acid residue substitutions, deletions or additions, by the covalent attachment of any type of molecule to the polypeptide (e.g., by glycosylation, acetylation, pegylation, phosphorylation, amidation, derivatization by known protecting/blocking groups, proteolytic cleavage, linkage to a cellular ligand or other protein). A derivative of an engineered antibody or antigen-binding fragment thereof can be modified by chemical modifications using techniques known to those of skill in the art, including, but not limited to, specific chemical cleavage, acetylation, formylation. Further, a derivative of an engineered antibody or antigen-binding fragment thereof can contain one or more non-classical amino acids. Typically, a polypeptide derivative possesses a similar or identical function as an engineered antibody or antigen-binding fragment thereof provided herein.

As used herein, the phrase “derived from” when referring to antibody fragments derived from another antibody, such as a monoclonal antibody, refers to the engineering of antibody fragments (e.g., Fab, F(ab′), F(ab′)2, single-chain Fv (scFv), Fv, dsFv, diabody, Fd and Fd′ fragments) that retain the binding specificity of the original antibody. Such fragments can be derived by a variety of methods known in the art, including, but not limited to, enzymatic cleavage, chemical crosslinking, recombinant means or combinations thereof. Generally, the derived antibody fragment shares the identical or substantially identical heavy chain variable region (VH) and light chain variable region (VL) of the parent antibody, such that the antibody fragment and the parent antibody bind the same epitope

As used herein, a “parent antibody” or “source antibody” refers the to an antibody from which an antibody fragment (e.g., Fab, F(ab′), F(ab′)2, single-chain Fv (scFv), Fv, dsFv, diabody, Fd and Fd′ fragments) is derived.

As used herein, the term “epitope” refers to any antigenic determinant on an antigen to which the paratope of an antibody binds. Epitopic determinants typically contain chemically active surface groupings of molecules such as amino acids or sugar side chains and typically have specific three-dimensional structural characteristics, as well as specific charge characteristics.

As used herein, a chimeric polypeptide refers to a polypeptide that contains portions from at least two different polypeptides or from two non-contiguous portions of a single polypeptide. Thus, a chimeric polypeptide generally includes a sequence of amino acid residues from all or part of one polypeptide and a sequence of amino acids from all or part of another different polypeptide. The two portions can be linked directly or indirectly and can be linked via peptide bonds, other covalent bonds or other non-covalent interactions of sufficient strength to maintain the integrity of a substantial portion of the chimeric polypeptide under equilibrium conditions and physiologic conditions, such as in isotonic pH 7 buffered saline. For purposes herein, chimeric polypeptides include those containing all or part of an engineered antibody linked to another polypeptide, such as, for example, a multimerization domain, a heterologous immunoglobulin constant domain or framework region, or a diagnostic or therapeutic polypeptide.

As used herein, a fusion protein is a polypeptide engineered to contain sequences of amino acids corresponding to two distinct polypeptides, which are joined together, such as by expressing the fusion protein from a vector containing two nucleic acids, encoding the two polypeptides, in close proximity, e.g., adjacent, to one another along the length of the vector. Generally, a fusion protein provided herein refers to a polypeptide that contains a polypeptide having the amino acid sequence of an antibody or antigen-binding fragment thereof and a polypeptide or peptide having the amino acid sequence of a heterologous polypeptide or peptide, such as, for example, a diagnostic or therapeutic polypeptide. Accordingly, a fusion protein refers to a chimeric protein containing two, or portions from two, or more proteins or peptides that are linked directly or indirectly via peptide bonds. The two molecules can be adjacent in the construct or separated by a linker, or spacer polypeptide. The spacer can encode a polypeptide that alters the properties of the polypeptide, such as solubility or intracellular trafficking.

As used herein, “linker” or “spacer” peptide refers to short sequences of amino acids that join two polypeptide sequences (or nucleic acid encoding such an amino acid sequence). “Peptide linker” refers to the short sequence of amino acids joining the two polypeptide sequences. Exemplary of polypeptide linkers are linkers joining a peptide transduction domain to an antibody or linkers joining two antibody chains in a synthetic antibody fragment such as an scFv fragment. Linkers are well-known and any known linkers can be used in the provided methods. Exemplary of polypeptide linkers are (Gly-Ser) amino acid sequences, with some Glu or Lys residues dispersed throughout to increase solubility. Other exemplary linkers are described herein; any of these and other known linkers can be used with the provided compositions and methods.

As used herein, “antibody hinge region” or “hinge region” refers to a polypeptide region that exists naturally in the heavy chain of the gamma, delta and alpha antibody isotypes, between the CH1 and CH2 domains that has no homology with the other antibody domains. This region is rich in proline residues and gives the IgG, IgD and IgA antibodies flexibility, allowing the two “arms” (each containing one antibody combining site) of the Fab portion to be mobile, assuming various angles with respect to one another as they bind antigen. This flexibility allows the Fab arms to move in order to align the antibody combining sites to interact with epitopes on cell surfaces or other antigens. Two interchain disulfide bonds within the hinge region stabilize the interaction between the two heavy chains. In some embodiments provided herein, the synthetically produced antibody fragments contain one or more hinge regions, for example, to promote stability via interactions between two antibody chains. Hinge regions are exemplary of dimerization domains.

As used herein, “humanized antibodies” refer to antibodies that are modified to include “human” sequences of amino acids so that administration to a human does not provoke an immune response. A humanized antibody typically contains complementarily determining regions (CDRs) derived from a non-human species immunoglobulin and the remainder of the antibody molecule derived mainly from a human immunoglobulin. Methods for preparation of such antibodies are known. For example, DNA encoding a monoclonal antibody can be altered by recombinant DNA techniques to encode an antibody in which the amino acid composition of the non-variable regions is based on human antibodies. Methods for identifying such regions are known, including computer programs, which are designed for identifying the variable and non-variable regions of immunoglobulins.

As used herein, an “Ig domain” is a domain, recognized as such by those in the art, that is distinguished by a structure, called the Immunoglobulin (Ig) fold, which contains two beta-pleated sheets, each containing anti-parallel beta strands of amino acids connected by loops. The two beta sheets in the Ig fold are sandwiched together by hydrophobic interactions and a conserved intra-chain disulfide bond. Individual immunoglobulin domains within an antibody chain further can be distinguished based on function. For example, a light chain contains one variable region domain (VL) and one constant region domain (CL), while a heavy chain contains one variable region domain (VH) and three or four constant region domains (CH). Each VL, CL, VH, and CH domain is an example of an immunoglobulin domain.

As used herein, a “variable domain” or “variable region” is a specific Ig domain of an antibody heavy or light chain that contains a sequence of amino acids that varies among different antibodies. Each light chain and each heavy chain has one variable region domain, VL and VH, respectively. The variable domains provide antigen specificity, and thus are responsible for antigen recognition. Each variable region contains CDRs that are part of the antigen-binding site domain and framework regions (FRs).

As used herein, “antigen-binding domain,” “antigen-binding site,” “antigen combining site” and “antibody combining site” are used synonymously to refer to a domain within an antibody that recognizes and physically interacts with cognate antigen. A native conventional full-length antibody molecule has two conventional antigen-binding sites, each containing portions of a heavy chain variable region and portions of a light chain variable region. A conventional antigen-binding site contains the loops that connect the anti-parallel beta strands within the variable region domains. The antigen combining sites can contain other portions of the variable region domains. Each conventional antigen-binding site contains three hypervariable regions from the heavy chain and three hypervariable regions from the light chain. The hypervariable regions also are called complementarity-determining regions (CDRs).

As used herein, “hypervariable region,” “HV,” “complementarity-determining region” and “CDR” and “antibody CDR” are used interchangeably to refer to one of a plurality of portions within each variable region that together form an antigen-binding site of an antibody. Each variable region domain contains three CDRs, named CDR1, CDR2 and CDR3. The three CDRs are non-contiguous along the linear amino acid sequence but are proximate in the folded polypeptide. The CDRs are located within the loops that join the parallel strands of the beta sheets of the variable domain. As described herein, one of skill in the art knows and can identify the CDRs and framework regions based on Kabat or Chothia numbering (see e.g., Kabat, E. A. et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, the disclosures of which are incorporated by reference herein).

As used herein, “framework regions” (FRs) are the domains within the antibody variable region domains that are located within the beta sheets; the FR regions are comparatively more conserved, in terms of their amino acid sequences, than the hypervariable regions.

As used herein, a “constant region” or “constant domain” is a domain in an antibody heavy or light chain that contains a sequence of amino acids that is comparatively more conserved than that of the variable region domain. In conventional full-length antibody molecules, each light chain has a single light chain constant region (CL) domain and each heavy chain contains one or more heavy chain constant region (CH) domains, which include, CH1, CH2, CH3 and CH4. Full-length IgA, IgD and IgG isotypes contain CH1, CH2, CH3 and a hinge region, while IgE and IgM contain CH1, CH2, CH3 and CH4. CH1 and CL domains extend the Fab arm of the antibody molecule, thus contributing to the interaction with antigen and rotation of the antibody arms. Antibody constant regions can serve effector functions, such as, but not limited to, clearance of antigens, pathogens and toxins to which the antibody specifically binds, e.g., through interactions with various cells, biomolecules and tissues.

As used herein, a functional region of an antibody is a portion of the antibody that contains at least a VH, VL, CH (e.g. CH1, CH2 or CH3), CL or hinge region domain of the antibody, or at least a functional region thereof

As used herein, “specifically bind” or “immunospecifically bind” with respect to an antibody or antigen-binding fragment thereof are used interchangeably herein and refer to the ability of the antibody or antigen-binding fragment to form one or more noncovalent bonds with a cognate antigen, by noncovalent interactions between the antibody combining site(s) of the antibody and the antigen. Affinity constants can be determined by standard kinetic methodology for antibody reactions, for example, immunoassays, surface plasmon resonance (SPR) (Rich and Myszka (2000) Curr. Opin. Biotechnol 11:54; Englebienne (1998) Analyst. 123:1599), isothermal titration calorimetry (ITC) or other kinetic interaction assays known in the art (see, e.g., Paul, ed., Fundamental Immunology, 2nd ed., Raven Press, New York, pages 332-336 (1989)). Instrumentation and methods for real time detection and monitoring of binding rates are known and are commercially available (e.g., BiaCore 2000, Biacore AB, Upsala, Sweden and GE Healthcare Life Sciences; Malmqvist (2000) Biochem. Soc. Trans. 27:335). Typically, an antibody or antigen-binding fragment thereof provided herein that binds immunospecifically to an epitope does not cross-react with other antigens or cross reacts with substantially (at least 10-100 fold) lower affinity for such antigens. Antibodies or antigen-binding fragments that immunospecifically bind to a particular epitope can be identified, for example, by immunoassays, such as radioimmunoassays (RIA), enzyme-linked immunosorbent assays (ELISAs), surface plasmon resonance, or other techniques known to those of skill in the art. An antibody or antigen-binding fragment thereof that immunospecifically binds to an epitope typically is one that binds to the epitope with a higher binding affinity than to any cross-reactive epitope as determined using experimental techniques, such as, but not limited to, immunoassays, surface plasmon resonance, or other techniques known to those of skill in the art. Immunospecific binding to an isolated protein (i.e., a recombinantly produced protein), does not necessarily mean that the antibody will exhibit the same immunospecific binding. Such measurements and properties are distinct. The affinity for the antibody or antigen-binding fragments for the antigen as presented can be determined. For purposes herein, when describing an affinity or related term, the target, such as the isolated protein, will be identified.

As used herein, “Fc” or “Fc region” or “Fc domain” refers to a polypeptide containing the constant region of an antibody heavy chain, excluding the first constant region immunoglobulin domain. Thus, Fc refers to the last two constant region immunoglobulin domains of IgA, IgD, and IgE, or the last three constant region immunoglobulin domains of IgE and IgM. Optionally, an Fc domain can include all or part of the flexible hinge N-terminal to these domains. For IgA and IgM, Fc can include the J chain. For an exemplary Fc domain of IgG, Fc contains immunoglobulin domains Cγ2 and Cγ3, and optionally, all or part of the hinge between Cγ1 and Cγ2. The boundaries of the Fc region can vary, but typically, include at least part of the hinge region. In addition, Fc also includes any allelic or species variant or any variant or modified form, such as any variant or modified form that alters the binding to an FcR or alters an Fc-mediated effector function.

As used herein, a “tag” or an “epitope tag” refers to a sequence of amino acids, typically added to the N- or C-terminus of a polypeptide, such as an antibody provided herein. The inclusion of tags fused to a polypeptide can facilitate polypeptide purification and/or detection. Typically, a tag or tag polypeptide refers to polypeptide that has enough residues to provide an epitope recognized by an antibody or can serve for detection or purification yet is short enough such that it does not interfere with activity of chimeric polypeptide to which it is linked. The tag polypeptide typically is sufficiently unique so an antibody that specifically binds thereto does not substantially cross-react with epitopes in the polypeptide to which it is linked. Suitable tag polypeptides generally have at least 5 or 6 amino acid residues and usually between about 8-50 amino acid residues, typically between 9-30 residues. The tags can be linked to one or more chimeric polypeptides in a multimer and permit detection of the multimer or its recovery from a sample or mixture. Such tags are well known and can be readily synthesized and designed. Exemplary tag polypeptides include those used for affinity purification and include, His tags, the influenza hemagglutinin (HA) tag polypeptide and its antibody 12CA5, (Field et al. (1988) Mol. Cell. Biol. 8:2159-2165); the c-myc tag and the 8F9, 3C7, 6E10, G4, B7 and 9E10 antibodies thereto (see, e.g., Evan et al. (1985) Molecular and Cellular Biology 5 :3610-3616); and the Herpes Simplex virus glycoprotein D (gD) tag and its antibody (Paborsky et al. (1990) Protein Engineering 3:547-553 (1990). An antibody used to detect an epitope-tagged antibody is typically referred to herein as a secondary antibody.

As used herein, “polypeptide” refers to two or more amino acids covalently joined. The terms “polypeptide” and “protein” are used interchangeably herein.

As used herein, a “peptide” refers to a polypeptide that is from 2 to about or 40 amino acids in length.

As used herein, an “amino acid” is an organic compound containing an amino group and a carboxylic acid group. A polypeptide contains two or more amino acids. For purposes herein, amino acids contained in the antibodies provided include the twenty naturally-occurring amino acids, non-natural amino acids, and amino acid analogs (e.g., amino acids wherein the α-carbon has a side chain). As used herein, the amino acids, which occur in the various amino acid sequences of polypeptides appearing herein, are identified according to their well-known, three-letter or one-letter abbreviations. The nucleotides, which occur in the various nucleic acid molecules and fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3557-59 (1968) and adopted at 37 C.F.R. §§ 1.821-1.822, abbreviations for amino acid residues are used throughout. All sequences of amino acid residues represented herein by a formula have a left to right orientation in the conventional direction of amino-terminus to carboxyl-terminus. In addition, the phrase “amino acid residue” is defined to include natural, modified, non-natural and unusual amino acids. Furthermore, a dash at the beginning or end of an amino acid residue sequence indicates a peptide bond to a further sequence of one or more amino acid residues or to an amino-terminal group such as NH₂ or to a carboxyl-terminal group such as COOH. As used herein, “naturally occurring amino acids” refer to the 20 L-amino acids that occur in polypeptides.

Stability” and “stable” refer to the resistance of engineered antibodies in a formulation to aggregation, degradation or fragmentation under given manufacture, preparation, transportation and storage conditions. An engineered antibody with improved stability will retain biological activity under given manufacture, preparation, transportation and storage conditions. The stability of an engineered antibody can be assessed by degrees of aggregation, degradation or fragmentation, as measured by High Performance Size Exclusion Chromatography (HPSEC), static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques. The stability of an engineered antibody may be compared to a comparable molecule under identical conditions. The overall stability of an engineered antibody can also be assessed by various immunological assays including, for example, ELISA and radioimmunoassay using isolated antigen molecules or cells expressing the same.

As used herein, an “activity” or a “functional activity” of a polypeptide, such as an antibody, refers to any activity exhibited by the polypeptide. Such activities can be empirically determined. Exemplary activities include, but are not limited to, ability to interact with a biomolecule, for example, through antigen-binding, DNA binding, ligand binding, or dimerization, enzymatic activity, for example, kinase activity or proteolytic activity. For an antibody (including antibody fragments), activities include, but are not limited to, the ability to specifically bind a particular antigen, affinity of antigen-binding (e.g. high or low affinity), avidity of antigen-binding (e.g. high or low avidity), on-rate, off-rate, effector functions, such as the ability to promote antigen neutralization or clearance, virus neutralization, and in vivo activities, such as the ability to prevent infection or invasion of a pathogen, or to promote clearance, or to penetrate a particular tissue or fluid or cell in the body or improved manufacturability, thermostability, or protease resistance. Activity can be assessed in vitro or in vivo using recognized assays, such as ELISA, flow cytometry, surface plasmon resonance or equivalent assays to measure on- or off-rate, immunohistochemistry and immunofluorescence histology and microscopy, cell-based assays, flow cytometry and binding assays (e.g., panning assays). For example, for an antibody polypeptide, activities can be assessed by measuring binding affinities, avidities, and/or binding coefficients (e.g., for on-/off-rates), and other activities in vitro or by measuring various effects in vivo, such as immune effects, e.g. antigen clearance, penetration or localization of the antibody into tissues, protection from disease, e.g. infection, serum or other fluid antibody titers, or other assays that are well known in the art. The results of such assays that indicate that a polypeptide exhibits an activity can be correlated to activity of the polypeptide in vivo, in which in vivo activity can be referred to as therapeutic activity, or biological activity. Activity of a modified polypeptide can be any level of percentage of activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more of activity compared to the unmodified polypeptide. Assays to determine functionality or activity of modified (e.g. variant) antibodies are well known in the art.

As used herein, “exhibits at least one or more improved properties” refers to the activity exhibited by a modified polypeptide, such as a variant polypeptide produced according to the provided methods, such as a modified, e.g. variant antibody or other therapeutic polypeptide (e.g. a modified engineered antibody or antigen-binding fragment thereof), compared to the target or unmodified polypeptide, that does not contain the modification. A modified, or variant, polypeptide that retains an activity of a target polypeptide can exhibit improved activity (for example one or more of improved manufacturability, thermostability, or protease resistance) or maintain the activity of the unmodified polypeptide. In some instances, a modified, or variant, polypeptide can retain an activity that is increased compared to a target or unmodified polypeptide. In some cases, a modified, or variant, polypeptide can retain an activity that is decreased compared to an unmodified or target polypeptide. Activity of a modified, or variant, polypeptide can be any level of percentage of activity of the unmodified or target polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more activity compared to the unmodified or target polypeptide. In other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times greater than unmodified or target polypeptide. Assays for retention of an activity depend on the activity to be retained. Such assays can be performed in vitro or in vivo. Activity can be measured, for example, using assays known in the art and described in the Examples below for activities such as but not limited to ELISA and panning assays. Activities of a modified, or variant, polypeptide compared to an unmodified or target polypeptide also can be assessed in terms of an in vivo therapeutic or biological activity or result following administration of the polypeptide.

As used herein, “nucleic acid” refers to at least two linked nucleotides or nucleotide derivatives, including a deoxyribonucleic acid (DNA) and a ribonucleic acid (RNA), joined together, typically by phosphodiester linkages. Also included in the term “nucleic acid” are analogs of nucleic acids such as peptide nucleic acid (PNA), phosphorothioate DNA, and other such analogs and derivatives or combinations thereof. Nucleic acids also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). The term also includes, as equivalents, derivatives, variants and analogs of either RNA or DNA made from nucleotide analogs, single (sense or antisense) and double-stranded nucleic acids. Deoxyribonucleotides include deoxyadenosine, deoxycytidine, deoxyguanosine and deoxythymidine. For RNA, the uracil base is uridine.

As used herein, “regulatory region” of a nucleic acid molecule means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, “operably linked” with reference to nucleic acid sequences, regions, elements or domains means that the nucleic acid regions are functionally related to each other. For example, nucleic acid encoding a leader peptide can be operably linked to nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, “production by recombinant means by using recombinant DNA methods” means the use of the well-known methods of molecular biology for expressing proteins encoded by cloned DNA.

As used herein, “expression” refers to the process by which polypeptides are produced by transcription and translation of polynucleotides. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and Bradford protein assay.

As used herein, a “host cell” is a cell that is used in to receive, maintain, reproduce and amplify a vector. A host cell also can be used to express the polypeptide encoded by the vector. The nucleic acid contained in the vector is replicated when the host cell divides, thereby amplifying the nucleic acids. In one example, the host cell is a genetic package, which can be induced to express the variant polypeptide on its surface. In another example, the host cell is infected with the genetic package. For example, the host cells can be phage-display compatible host cells, which can be transformed with phage or phagemid vectors and accommodate the packaging of phage expressing fusion proteins containing the variant polypeptides.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “similarity” between two proteins or nucleic acids refers to the relatedness between the sequence of amino acids of the proteins or the nucleotide sequences of the nucleic acids. Similarity can be based on the degree of identity of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

As used herein, when a polypeptide or nucleic acid molecule or region thereof contains or has “identity” or “homology” to another polypeptide or nucleic acid molecule or region, the two molecules and/or regions share greater than or equal to at or about 40% sequence identity, and typically greater than or equal to at or about 50% sequence identity, such as at least or about 60%, 65%, 70%, 75%, 80%, 85%, 90%, 95%, 96%, 97%, 98%, 99% or 100% sequence identity; the precise percentage of identity can be specified if necessary. A nucleic acid molecule, or region thereof, that is identical or homologous to a second nucleic acid molecule or region can specifically hybridize to a nucleic acid molecule or region that is 100% complementary to the second nucleic acid molecule or region. Identity alternatively can be compared between two theoretical nucleotide or amino acid sequences or between a nucleic acid or polypeptide molecule and a theoretical sequence.

Sequence “identity,” per se, has an art-recognized meaning and the percentage of sequence identity between two nucleic acid or polypeptide molecules or regions can be calculated using published techniques. Sequence identity can be measured along the full length of a polynucleotide or polypeptide or along a region of the molecule. (See, e.g.: Computational Molecular Biology, Lesk, A. M., ed., Oxford University Press, New York, 1988; Biocomputing: Informatics and Genome Projects, Smith, D.W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptides, the term “identity” is well known to skilled artisans (Carrillo, H. & Lipman, D., SIAM J Applied Math 48:1073 (1988)).

Sequence identity compared along the full length of two polynucleotides or polypeptides refers to the percentage of identical nucleotide or amino acid residues along the full-length of the molecule. For example, if a polypeptide A has 100 amino acids and polypeptide B has 95 amino acids, which are identical to amino acids 1-95 of polypeptide A, then polypeptide B has 95% identity when sequence identity is compared along the full length of a polypeptide A compared to full length of polypeptide B. Alternatively, sequence identity between polypeptide A and polypeptide B can be compared along a region, such as a 20 amino acid analogous region, of each polypeptide. In this case, if polypeptide A and B have 20 identical amino acids along that region, the sequence identity for the regions is 100%. Alternatively, sequence identity can be compared along the length of a molecule, compared to a region of another molecule. Alternatively, sequence identity between polypeptide A and polypeptide B can be compared along the same length polypeptide but with amino acid replacements, such as conservative amino acid replacements or non-conservative amino acid replacements. As discussed below, and known to those of skill in the art, various programs and methods for assessing identity are known to those of skill in the art. High levels of identity, such as 90% or 95% identity, readily can be determined without software.

Whether any two nucleic acid or polypeptide molecules have nucleotide sequences that are at least or about 60%, 70%, 80%, 85%, 90%, 95%, 96%, 97%, 98% or 99% “identical” can be determined using known computer algorithms such as the “FASTA” program, using for example, the default parameters as in Pearson et al. (1988) Proc. Natl. Acad. Sci. USA 85:2444 (other programs include the GCG program package (Devereux, J. et al. (1984) Nucleic Acids Research 12(I):387), BLASTP, BLASTN, FASTA (Altschul, S.F. et al. (1990) J. Molec. Biol. 215:403; Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

As used herein, a “modification” is in reference to modification of a sequence of amino acids of a polypeptide or a sequence of nucleotides in a nucleic acid molecule and includes deletions, insertions, and replacements of amino acids and nucleotides, respectively. Methods of modifying a polypeptide are routine to those of skill in the art, such as by using recombinant DNA methodologies.

As used herein, “substitution” refers to the replacing of one or more nucleotides or amino acids in a native, target, wild-type or other nucleic acid or polypeptide sequence with an alternative nucleotide or amino acid, without changing the length (as described in numbers of residues) of the molecule. Thus, one or more substitutions in a molecule does not change the number of amino acid residues or nucleotides of the molecule. Substitution mutations compared to a particular polypeptide can be expressed in terms of the number of the amino acid residue along the length of the polypeptide sequence.

As used herein, the phase “having the same binding specificity” when used to describe an antibody in reference to another antibody, means that the antibody specifically binds to all or a part of the same antigenic epitope as the reference antibody. The epitope can be in the isolated protein. The ability of two antibodies to bind to the same epitope can be determined by known assays in the art such as, for example, surface plasmon resonance assays and antibody competition assays. Typically, antibodies that immunospecifically bind to the same epitope can compete for binding to the epitope, which can be measured, for example, by an in vitro binding competition assay (e.g. competition ELISA), using techniques known the art. Typically, a first antibody that immunospecifically binds to the same epitope as a second antibody can compete for binding to the epitope by about or 30%, 35%, 40%, 45%, 50%, 55%, 60%, 65%, 70%, 75%, 80%, 85%, 90%, 95%, 100%, where the percentage competition is measured ability of the second antibody to displace binding of the first antibody to the epitope. In exemplary competition assays, the antigen is incubated in the presence a predetermined limiting dilution of a labeled antibody (e.g., 50-70% saturation concentration), and serial dilutions of an unlabeled competing antibody. Competition is determined by measuring the binding of the labeled antibody to the antigen for any decreases in binding in the presence of the competing antibody. Variations of such assays, including various labeling techniques and detection methods including, for example, radiometric, fluorescent, enzymatic and colorimetric detection, are known in the art.

As used herein, “disease or disorder” refers to a pathological condition in an organism resulting from cause or condition including, but not limited to, infections, acquired conditions, genetic conditions, and characterized by identifiable symptoms.

As used herein, “treating” a subject with a disease or condition means that the subject's symptoms are partially or totally alleviated or remain static following treatment. Hence treatment encompasses prophylaxis, therapy and/or cure. Prophylaxis refers to prevention of a potential disease and/or a prevention of worsening of symptoms or progression of a disease. Treatment also encompasses any pharmaceutical use of any antibody or antigen-binding fragment thereof provided or compositions provided herein.

As used herein, “prevention” or prophylaxis, and grammatically equivalent forms thereof, refers to methods in which the risk of developing disease or condition is reduced.

As used herein, a “pharmaceutically effective agent” includes any therapeutic agent or bioactive agents, including, but not limited to, for example, anesthetics, vasoconstrictors, dispersing agents, conventional therapeutic drugs, including small molecule drugs and therapeutic proteins.

As used herein, a “therapeutic effect” means an effect resulting from treatment of a subject that alters, typically improves or ameliorates the symptoms of a disease or condition or that cures a disease or condition.

As used herein, a “therapeutically effective amount” or a “therapeutically effective dose” refers to the quantity of an agent, compound, material, or composition containing a compound that is at least sufficient to produce a therapeutic effect following administration to a subject. Hence, it is the quantity necessary for preventing, curing, ameliorating, arresting or partially arresting a symptom of a disease or disorder.

As used herein, “therapeutic efficacy” refers to the ability of an agent, compound, material, or composition containing a compound to produce a therapeutic effect in a subject to whom an agent, compound, material, or composition containing a compound has been administered.

As used herein, a “prophylactically effective amount” or a “prophylactically effective dose” refers to the quantity of an agent, compound, material, or composition containing a compound that when administered to a subject, will have the intended prophylactic effect, e.g., preventing or delaying the onset, or reoccurrence, of disease or symptoms, reducing the likelihood of the onset, or reoccurrence, of disease or symptoms, or reducing the incidence of viral infection. The full prophylactic effect does not necessarily occur by administration of one dose and can occur only after administration of a series of doses. Thus, a prophylactically effective amount can be administered in one or more administrations.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, a “label” or “detectable moiety” is a detectable marker (e.g., a fluorescent molecule, chemiluminescent molecule, a bioluminescent molecule, a contrast agent (e.g., a metal), a radionuclide, a chromophore, a detectable peptide, or an enzyme that catalyzes the formation of a detectable product) that can be attached or linked directly or indirectly to a molecule or associated therewith and can be detected in vivo and/or in vitro. The detection method can be any method known in the art, including known in vivo and/or in vitro methods of detection (e.g., imaging by visual inspection, magnetic resonance (MR) spectroscopy, ultrasound signal, X-ray, gamma ray spectroscopy (e.g., positron emission tomography (PET) scanning, single-photon emission computed tomography (SPECT)), fluorescence spectroscopy or absorption). Indirect detection refers to measurement of a physical phenomenon, such as energy or particle emission or absorption, of an atom, molecule or composition that binds directly or indirectly to the detectable moiety (e.g., detection of a labeled secondary antibody or antigen-binding fragment thereof that binds to a primary antibody.

As used herein, the term “individual” or “subject” is used interchangeably to refer to an animal, including a mammal, such as a human being.

As used herein, “animal” includes any animal, such as, but are not limited to primates including humans, gorillas and monkeys; rodents, such as mice and rats; fowl, such as chickens; ruminants, such as goats, cows, deer, sheep; pigs and other animals. Non-human animals exclude humans as the contemplated animal.

As used herein, an “isolated” or “purified” polypeptide or protein (e.g. an isolated antibody or antigen-binding fragment thereof) or biologically-active portion thereof (e.g. an isolated antigen-binding fragment) is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification does not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound. As used herein, a “cellular extract” or “lysate” refers to a preparation or fraction which is made from a lysed or disrupted cell.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

As used herein, “optional” or “optionally” means that the subsequently circumstance or limitation on scope does or does not occur, and that the description includes instances where the circumstance or limitation on scope occurs and instances where it does not. For example, an a composition that optionally contains additional exogenous enzymes means that the enzymes can be present or not present in the composition.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Compositions

A. Engineered Antibodies

Provided herein are non-naturally occurring modified antibodies, fragments thereof, or variants thereof with modified framework and/or constant regions having improved expression, resistance to cleavage (such as proteolytic cleavage), and/or thermostability properties. Said modifications of the antibody and the modified antibodies disclosed are referred to herein as “engineered antibodies.” The engineered antibodies can exhibit alterations in one or more of the characteristics of the antibody, including, but not limited to, improved manufacturability, stability (such as thermostability), flexibility, length, conformation, charge, resistance to cleavage (such as proteolytic cleavage), hydrophobicity, and decreased aggregation, relative to a wild type (i.e. parent or unmodified) antibody. The engineered antibodies disclosed herein may be generated by methods well known in the art. Substitutions which may be utilized to generate an engineered antibody include, but are not limited to, amino acid insertions, deletions, substitutions, and rearrangements.

In some embodiments, provided herein are isolated monoclonal antibodies or functional fragment thereof comprising: (a) a heavy chain variable region comprising one or more amino acid substitution(s) (such as any of 1, 2, 3, or 4 amino acid substitutions) comprising T or an I at Kabat position 5; N at Kabat position 50, V or Y at Kabat position 84; and/or S at Kabat position 85; and or (b) a heavy chain constant region comprising one or more amino acid substitution(s) (such as any of 1, 2, 3, 4, 5, 6, or 7 amino acid substitutions) comprising Q at position 17; V at position 18; V at position 64; L at position 151; C, E, or D at position 152; N at position 156; and/or P at position 157, wherein the amino acid positions of the heavy chain constant region correspond to those of SEQ ID NO:1, The engineered antibody can differ by at least one amino acid (such as any of 1, 2, 3, 4, 5, 6, 7, 8, 9, 10, or 11 amino acid substitutions) from a parent antibody. Additionally, the engineered antibody can exhibit one or more improved properties including, but not limited to, increased manufacturability, thermostability, and/or protease resistance compared to an unmodified parent antibody.

The engineered antibody or functional fragment thereof can comprise or further comprise (c) a light chain variable region comprising an amino acid substitution comprising F at Kabat position 42; and/or (d) a light chain constant region comprising one or more amino acid substitution(s) (such as any of 1, 2, or 3 amino acid substitutions) comprising T at position 81; M or V at position 97; and/or I at position 100, wherein the amino acid positions of the light chain constant region correspond to those of SEQ ID NO:2.

In some embodiments, the antibody exhibits increased or improved manufacturability compared to a parent antibody that has not been so engineered. For example, the engineered antibody may display reduced aggregation-propensity, and/or increased productivity upon expression, relative to the parent antibody. For example, the engineered antibody may display an increase in productivity of at least 10%, at least 20%, at least 30%, at least 40%, at least 50%, at least 100%, at least 200%, or at least 500% relative to the parent antibody and/or a decrease in aggregation (i.e. a reduction in the proportion of molecules in the native state ensemble which are aggregated) of at least 10%, at least 20%, at least 30%, at least 40%, at least 50%, at least 80%, at least 90% or at least 99% relative to the parent antibody. The engineered antibody can display a decrease in aggregation of up to 100% relative to the parent immunoglobulin (i.e. complete abolition of aggregation).

Improvements in manufacturability may result fully or partially from reduced aggregation-propensity relative to the parent immunoglobulin. “Aggregation-propensity” relates to the tendency of the engineered antibody to form insoluble aggregates after expression in a recombinant system. Reductions in aggregation propensity reduce the proportion of molecules in the native state ensemble of the immunoglobulin which exist in an aggregated form (Carpenter et al, 2009 J Pharm Sci. April; 98(4):1201-5). In other words, the proportion of molecules within the native state ensemble of the engineered antibody which exist in an aggregated or insoluble form is lower than the proportion within the native state ensemble of the parent antibody.

An engineered antibody disclosed herein may show less self-association or aggregation compared to a parent antibody either under native conditions or at increased temperature (e.g. 60° C.) (i.e. conditions under which the antigen binding site of an immunoglobulin does not unfold). Preferably, the engineered antibody shows less self-association or aggregation than parent antibody under native conditions (e.g. conditions which do not lead to unfolding of the antibody).

Aggregation propensity as described herein is distinct from thermal refolding efficiency (TRE), which relates to the ability of a protein to correctly refold after thermal denaturation and is typically measured using circular dichroism (CD) (Tanha et al Protein Eng Des Sel. 2006 November; 19(11):503-9). Reductions in the aggregation propensity of an antibody as described herein may have little or no effect on the thermal refolding efficiency of the immunoglobulin. Thermal refolding efficiency is therefore independent of aggregation propensity and does not have a significant impact on the manufacturability of an antibody.

Aggregation may be measured by conventional methods. Suitable techniques include GP-HPLC, HPLC and AUC (Gabrielson J P et al J Pharm Sci 2007 96(2): 268-79), protein loss after filtration; turbidity; fluorescent dye binding (e.g. Nile Red, thioflavin T or 8-anilino-1-napthalenesulfonic acid; see for example Hawe, A. et al Pharmaceutical Research 2008 25 (7) 1487-99 or Demeule, B et al 2007 Int J Pharm 329: 37-45), field-flow fractionation (FFF; Demeule, B et al. mAbs 2009 1(2): 142-150), and analytical ultracentrifugation (AU/AUC; Liu J et cal. AAPS J 2006 8: 580-9). Other suitable methods are described in Arvinte T. In “Methods for structural analysis of protein pharmaceuticals” AAPS Press, 2005: 661-6 and Kiese S et al J Pharm Sci 2008 97(10): 4347-66.

Improvements in manufacturability may result fully or partially from increased productivity relative to the parent antibody. An engineered antibody disclosed herein may display increased productivity compared to the non-engineered parent antibody. For example, the variant immunoglobulin may show increased yields or titers compared to a parent immunoglobulin when expressed in a recombinant system, e.g. bacterial or mammalian cells. Productivity may be measured using standard techniques, such as the Bradford assay, spectrophotometry and ELISA.

An engineered antibody disclosed herein may also display one or more of improved purification yields; reduced formulation problems; reduced immunogenicity and increased bioavailability relative to the parent antibody. In some embodiments, improvements in manufacturability may result from both reduced aggregation-propensity and increased productivity relative to the parent antibody. In some embodiments, an engineered antibody disclosed herein displays the same or substantially the same activity as the parent antibody (i.e. antigen binding activity).

In some embodiments, the engineered antibody exhibits increased or improved or enhanced thermostability compared to a non-engineered parent antibody. As used herein, “thermostabilized” or “thermostability” refers to the quality of a protein or antibody to resist chemical or physical change as a result of increasing temperature. For the purposes of this invention, alterations to the amino acid sequence of an antibody may be made to increase the thermostability of said antibody compared to the parent antibody. Thermostability may be determined by any known method in the field, including the measurement of the antibody melting temperature (Tm). Improvements in thermostability include increases in the Tm by greater than or equal to 0.1° C. to greater than or equal to 10.0° C. For example, the engineered antibody may display increased thermostability, relative to the parent antibody. For example, the engineered antibody may display an increase in thermostability of at least 10%, at least 20%, at least 30%, at least 40%, at least 50%, at least 100%, at least 200%, or at least 500% relative to the parent antibody.

In some embodiments, the engineered antibody exhibits increased or improved or enhanced protease resistance compared to a parent antibody. The term “protease resistance” refers to the ability of a molecule comprised of peptide bonds, to resist hydrolytic cleavage of one or more of its peptide bonds in the presence of a proteolytic enzyme. The resistance to proteolytic enzymes is a relative property and is compared to a molecule (such as a non-engineered parent antibody) which is less able to withstand hydrolytic cleavage of one or more of its peptide bonds over a specified time period and under specified conditions, including the pH and or temperature at which the cleavage resistance is tested. One result of proteolytic cleavage indicative that cleavage has occurred is the generation of smaller fragments (lower molecular weight) as compared to the molecular weight of the intact, non-cleaved parent molecule. An engineered antibody or a fragment thereof disclosed herein comprising a hinge, a CH2 domain and a CH3 domain is “protease resistant” or “resistant to proteolysis” or has “increased resistance to proteolysis” when more than about 60%, 65%, 70%, 75%, 80%, 85%, 90%, 95%, 96%, 97%, 98%, 99% or 100% of a full length antibody (such as any of the engineered antibodies disclosed herein) remains intact for a given period of time (such as about 1, 2, 3, 4, 5, 6, 7, 8, 9, 10, 11, 12, 13, 14, 15, 16, 17, 18, 19, 20, 21, 22, 23, or 24 hours) when digested by a protease (such as, but not limited to, pepsin, matrix metalloprotease-3 (MMP-3), matrix metalloprotease-12 (MMP-12), pepsin, glutamyl endopeptidase V8 of Staphylococcus aureus (GluV8), or immunoglobulin degrading enzyme of Streptococcus pyogenes (IdeS)) in a given buffer (e.g., Tris-buffered saline) at a given temperature (e.g., at 37° C.) at a given pH (e.g., at pH 7.5) at a given antibody concentration (e.g. of 0.5 mg/ml) with a given protease concentration (such as about approximately 1-2% (w/w) ratio to IgG). Amount of intact IgG can be assessed by SDS-PAGE.

B. Modification of Engineered Antibodies

The engineered antibodies or antigen-binding fragments thereof provided herein can be further modified. Modifications of an engineered antibody or antigen-binding fragment can improve one or more properties of the antibody, including, but not limited to, decreasing the immunogenicity of the antibody or antigen-binding fragment, improving the half-life of the antibody or antigen-binding fragment, such as reducing the susceptibility to proteolysis and/or reducing susceptibility to oxidation, and altering or improving of the binding properties of the antibody or antigen-binding fragment thereof. Exemplary modifications include, but are not limited to, modifications of the primary amino acid sequence of the engineered antibody or antigen-binding fragment thereof and alteration of the post-translational modification of the engineered antibody or antigen-binding fragment thereof. Exemplary post-translational modifications include, for example, glycosylation, acetylation, pegylation, phosphorylation, amidation, derivatization with protecting/blocking group, proteolytic cleavage, linkage to a cellular ligand or other protein. Other exemplary modifications include attachment of one or more heterologous peptides to the engineered antibody or antigen-binding fragment to alter or improve one or more properties of the antibody or antigen-binding fragment thereof.

1. Signal Sequences

In some embodiments, the engineered antibodies disclosed herein can include a signal sequence. The signal sequence can be any signal sequence that facilitates protein secretion from a host cell (e.g., a filamentous fungal host cell). In particular embodiments, the engineered antibody can comprise a signal sequence for a protein that is known to be highly secreted from a host cell in which the fusion protein is to be produced. The signal sequence employed can be endogenous or non-endogenous to the host cell in which the engineered antibody is to be produced.

Suitable signal sequences are known in the art (see, e.g., Ward et al, Bio/Technology 1990 8:435-440; and Paloheimo et al, Applied and Environmental Microbiology 2003 69: 7073-7082). Non-limiting examples of suitable signal sequences include those of cellobiohydrolase I, cellobiohydrolase II, endoglucanases I, II and III, α-amylase, aspartyl proteases, glucoamylase, phytase, mannanase, α and β glucosidases, bovine chymosin, human interferon and human tissue plasminogen activator and synthetic consensus eukaryotic signal sequences such as those described by Gwynne et al., (1987) Bio/Technology 5:713-719.

In some embodiments, if Trichoderma (e.g. T. reesei) is employed as a host cell, the signal sequence or carrier of T. reesei mannanase I (Man5A, or MANI), T. reesei cellobiohydrolase II (Cel6A or CBHII), endoglucanase I (Cel7b or EGI), endoglucanase II (Cel5a or EGII), endoglucanase III (Cel12A or EGIII), xylanases I or II (XynIIa or XynIIb) or T. reesei cellobiohydrolase I (Cel7a or CBHI) can be employed in the engineered antibody.

In other embodiments, if an Aspergillus (e.g. A. niger) is employed as a host cell, the signal sequence or carrier of A. niger glucoamylase (GlaA) or alpha amylase can be employed in the fusion polypeptide. Aspergillus niger and Aspergillus awamori glucoamylases have identical amino acid sequences. Two forms of the enzyme are generally recognized in culture supernatants. GAI is the full-length form (amino acid residues 1-616) and GAII is a natural proteolytic fragment comprising amino acid residues 1-512. GAI is known to fold as two separate domains joined by an extended linker region. The two domains are the 471-residue catalytic domain (amino acids 1-471) and the 108 residue starch binding domain (amino acids 509-616), the linker region between the two domains being 36 residues (amino acids 472-508). GAIT lacks the starch binding domain. Reference is made to Libby et al., (1994) Protein Engineering 7:1109-1114. In some embodiments, the glucoamylase which is used as a carrier protein and including a signal sequence will have greater than 95%, 96%, 97%, 98% and 99% sequence identity with a catalytic domain of an Aspergillus or Trichoderma glucoamylase. The term “catalytic domain” refers to a structural portion or region of the amino acid sequence of a protein which possess the catalytic activity of the protein.

2. Carriers

In particular embodiments, the signal sequence can comprise a “carrier” that contains the signal sequence at its N-terminus, where the carrier is at least an N-terminal portion of a protein that is endogenous to the cell and efficiently secreted by a cell. In certain embodiments, the signal sequence and the carrier protein are obtained from the same gene. In some embodiments, the signal sequence and the carrier protein are obtained from different genes.

The carrier protein can include all or part of the mature sequence of a secreted polypeptide. In some embodiments, full length secreted polypeptides are used. However, functional portions of secreted polypeptides can be employed. As used herein “portion” of a secreted polypeptide or grammatical equivalents means a truncated secreted polypeptide that retains its ability to fold into a normal, albeit truncated, configuration.

In some cases, the truncation of the secreted polypeptide means that the functional protein retains a biological function. In some embodiments, the catalytic domain of the secreted polypeptide is used, although other functional domains could be used, for example the substrate binding domain. In one embodiment, when glucoamylase is used as the carrier protein (e.g. glucoamylase from Aspergillus niger), functional portions retain the catalytic domain of the enzyme and include amino acids 1-471 (see, WO 03089614, e.g., Example 10, the disclosure of which is incorporated by reference herein). In another embodiment, when CBH I is used as the carrier protein (i.e. CBH I from Trichoderma reesei) functional portions retain the catalytic domain of the enzyme. Reference is made to SEQ ID NO:1 of FIG. 2 of WO 05093073, the disclosure of which is incorporated by reference herein, wherein the sequence encoding a Trichoderma reesei CBH1 signal sequence, T. reesei CBH1 catalytic domain (also referred to as catalytic core or core domain) and T. reesei CBH1 linker is disclosed. In some embodiments, a CBH1 carrier protein and including a signal sequence will have greater than 95%, 96%, 97%, 98% and 99% sequence identity with SEQ ID NO: 1 of FIG. 2 of WO 05093073, the disclosure of which is incorporated by reference herein).

In general, if the carrier protein is a truncated protein, it is C-terminally truncated (i.e., contains an intact N-terminus). Alternatively, the carrier protein can be N-terminally truncated, or optionally truncated at both ends to leave a functional portion. Generally, such portions of a secreted protein which comprise a carrier protein comprise greater than 50%, greater than 70%, greater than 80% and greater than 90% of the secreted protein and, in some embodiments, the N-terminal portion of the secreted protein. In some embodiments, the carrier protein will include a linker region in addition to the catalytic domain. In some embodiments, a portion of the linker region of the CBHI protein can be used in the carrier protein.

In some embodiments, the first amino acid sequence comprising a signal sequence functional as a secretory sequence is encoded by a first DNA molecule. The second amino acid sequence comprising the carrier protein is encoded by a second DNA sequence. However, as described above the signal sequence and the carrier protein can be obtained from the same gene.

3. Antibody Conjugates and Derivatives

Any of the engineered antibodies disclosed herein can include derivatives that are modified (i.e., by the covalent attachment of any type of molecule to the antibody such that covalent attachment). For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

Further, antibodies can be conjugated to albumin in order to make the antibody or antibody fragment more stable in vivo or have a longer half-life in vivo. The techniques are well known in the art, see e.g., International Publication Nos. WO 93/15199, WO 93/15200, and WO 01/77137; and European Patent No. EP 413, 622. The present invention encompasses the use of antibodies or fragments thereof conjugated or fused to one or more moieties, including but not limited to, peptides, polypeptides, proteins, fusion proteins, nucleic acid molecules, small molecules, mimetic agents, synthetic drugs, inorganic molecules, and organic molecules.

The present invention encompasses the use of antibodies or fragments thereof recombinantly fused or chemically conjugated (including both covalent and non-covalent conjugations) to a heterologous protein or polypeptide (or fragment thereof, for example, to a polypeptide of at least 10, at least 20, at least 30, at least 40, at least 50, at least 60, at least 70, at least 80, at least 90 or at least 100 amino acids) to generate fusion proteins. The fusion does not necessarily need to be direct but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International publication No. WO 93/21232; European Patent No. EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452.

The present invention further includes compositions comprising heterologous proteins, peptides or polypeptides fused or conjugated to antibody fragments. For exampl, the heterologous polypeptides may be fused or conjugated to a Fab fragment, Fd fragment, Fv fragment, F(ab)₂fragment, a VH domain, a VL domain, a VH CDR, a VL CDR, or fragment thereof Methods for fusing or conjugating polypeptides to antibody portions are well known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International publication Nos. WO 96/04388 and WO 91/06570; Ashkenazi et al., 1991, Proc. Natl. Acad. Sci. USA 88: 10535-10539; Meng et al., 1995, J. Immunoll. 154:5590-5600; and Vil et al., 1992, Proc. Natl. Acad. Sci. USA 89:11337-11341.

Additional fusion proteins, e.g., of antibodies that specifically bind an antigen (e.g., supra), may be generated through the techniques of gene-shuffling, motif-shuffling, exon-shuffling, and/or codon-shuffling (collectively referred to as “DNA shuffling”). DNA shuffling may be employed to alter the activities of the engineered antibodies disclosed herein or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997. Curr. Opinion Biotechnol. 8:724-33; Harayama, 1998, Trends Biotechnol. 16(2): 76-82; Hansson, 1999, J. Mol. Biol. 287:265-76; and Lorenzo and Blasco, 1998, Biotechniques 24(2): 308-313. Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions specifically bind to an Antigen may be recombined with one or more components, motifs, sections, parts, domains, fragments, etc. of one or more heterologous molecules.

Moreover, the antibodies or fragments thereof can be fused to marker sequences, such as a peptide to facilitate purification. In certain embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, the engineered antibodies disclosed herein, or analogs or derivatives thereof are conjugated to a, diagnostic or detectable agent. Such antibodies can be useful for monitoring or prognosing the development or progression of a cancer as part of a clinical testing procedure, such as determining the efficacy of a particular therapy. Such diagnosis and detection can be accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidinlbiotin and avidin/biotin.; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminal; bioluminescent materials, such as but not limited to, luciferase, luciferin, and aequorin; radioactive materials, such as but not limited to iodine (¹³¹I, ¹²⁵I, ¹²³I, ¹²¹I) carbon (¹⁴C), sulfur (³⁵S), tritium (³H), indium ¹¹⁵In, ¹¹³In, ¹¹²In, ¹¹¹In,), and and technetium (⁹⁹Tc), thallium (²⁰¹Ti), gallium (⁶⁸Ga, ⁶⁷Ga), palladium (¹⁰³Pd), molybdenum (⁹⁹Mo), xenon (₁₃₃Xe), fluorine (¹⁸F), ¹⁵³Sm, ¹⁷⁷Lu, ¹⁵⁹Gd, ¹⁴⁹Pm, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, noradioactive paramagnetic metal ions, and molecules that are radiolabelled or conjugated to specific radioisotopes.

Use of the engineered antibodies disclosed herein or fragments thereof conjugated to a therapeutic agent is also contemplated. An antibody or fragment thereof may be conjugated to a therapeutic moiety such as a cytotoxin, e.g., a cytostatic cytocidal agent, a therapeutic agent or a radioactive metal ion, e.g., alpha-emitters. A cytotoxin or cytotoxic agent includes any agent that is detrimental to cells. Examples include ribonuclease, monomethylauristatin E and F, paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g, dactinomycin (formerly actinomycin), bleomycin, mithramycin, and anthramycin (AMC)), and anti-mitotic agents (e.g., vincristine and vinblastine). A more extensive list of therapeutic moieties can be found in PCT publications WO 03/075957, incorporated by reference herein.

Further, an antibody or fragment thereof may he conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, fOr example, a toxin such as abrin, ricin A, Onconase (or another cytotoxic RNase), pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, an antibody can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (ROTA) which can be attached to the antibody via a linker molecule. Such linker molecules are commonly known in the art and described in Denardo et al., 1998, Clin Cancer. Res. 4:2483; Peterson et al., 1999, Bioconjug. Chem. 10:553; and Zimmerman et al., 1999, Nucl. Med. Biol. 26:943.

Techniques for conjugating therapeutic moieties to antibodies and related molecules are well known. Moieties can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv Drug Deliv Rev 53:171). Techniques for conjugating therapeutic moieties to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera. et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119.

Methods for fusing or conjugating antibodies and related molecules to polypeptide moieties are known in the art. See, e.g., U.S. Pat. Nos. 5,336,603; 5,622,929; 5,359,046; 5,349,053; 5,447,851, and 5,112,946; EP 307,434; EP 367,166; PCT Publications WO 96/04388 and WO 91/06570; Ashkenazi et al., 1991, PNAS USA 88:10535; Zheng et al., 1995, J Immunol 154:5590; and Vil et al., 1992, PNAS USA 89:11337. The fusion of an antibody to a moiety does not necessarily need to be direct but may occur through linker sequences. Such linker molecules are commonly known in the art and described in Denardo et al., 1998, Clin Cancer Res 4:2483; Peterson et al., 1999, Bioconjug Chem 10:553; Zimmerman et al., 1999, Nucl Med Biol 26:943; Garnett, 2002, Adv Drug Deliv Rev 53:171.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The therapeutic moiety or drug conjugated to an engineered antibody (such as any of those disclosed herein) should be chosen to achieve the desired prophylactic or therapeutic effect(s) for a particular disorder in a subject. A clinician or other medical personnel should consider the following when deciding on which therapeutic moiety or drug to conjugate to an engineered antibody: the nature of the disease, the severity of the disease, and the condition of the subject.

B. Polynucleotides

Another aspect of the compositions and methods disclosed herein is a polynucleotide or a nucleic acid sequence that encodes an engineered antibody, such as any of the engineered antibodies disclosed herein.

A fusion DNA construct encoding an engineered antibody as disclosed above is provided herein, comprising in operable linkage a promoter; a first DNA molecule encoding a signal sequence; a second DNA molecule encoding a carrier protein; a third DNA molecule encoding an antibody (e.g. a heavy chain and/or a light chain) or functional fragment thereof. The components of the fusion DNA construct can occur in any order. Since the genetic code is known, the design and production of these nucleic acids is well within the skill of an ordinarily skilled artisan, given the description of the engineered antibodies disclosed herein. In certain embodiments, the nucleic acids can be codon optimized for expression of the engineered antibodies in a particular host cell. Since codon usage tables are available for many species of, for example, mammalian cells and filamentous fungi, the design and production of codon-optimized nucleic acids that encodes subject engineered antibodies would be well within the skill of one of skill in the art.

C. Promoters

Examples of suitable promoters for directing the transcription of a nucleic acid in a host cell (for example, a filamentous fungal host cell) are promoters obtained from the genes for Aspergillus oryzae TAKA amylase, Rhizomucor miehei aspartic proteinase, Aspergillus niger neutral alpha-amylase, Aspergillus niger acid stable alpha-amylase (Korman et al (1990) Curr. Genet 17:203-212; Gines et al., (1989) Gene 79: 107-117), Aspergillus niger or Aspergillus awamori glucoamylase (glaA) (Nunberg et al., (1984) Mol. Cell Biol. 4:2306-2315; Boel E. et al., (1984) EMBO J. 3: 1581-1585), Rhizomucor miehei lipase, Aspergillus oryzae alkaline protease, Aspergillus oryzae triose phosphate isomerase, Aspergillus nidulans acetamidase (Hyner et al., (1983) Mol. Cell. Biol. 3:1430-1439), Fusarium venenatum amyloglucosidase, Fusarium oxysporum trypsin-like protease (WO 96/00787), Trichoderma reesei cellobiohydrolase I (Shoemaker et al. (1984) EPA EPO 0137280), Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof. Reference is also made to Yelton et al., (1984) Proc. Natl. Acad. Sci. USA 81:1470-1474; Mullaney et al., (1985) Mol. Gen. Genet. 199:37-45; Lockington et al., (1986) Gene 33: 137-149; Macknight et al., (1986) Cell 46: 143-147; Hynes et al., (1983) Mol. Cell Biol. 3: 1430-1439. Higher eukaryotic promoters such as SV40 early promoter (Barclay et al (1983) Molecular and Cellular Biology 3:2117-2130) can also be useful. Promoters can be constitutive or inducible promoters. Exemplary promoters include a Trichoderma reesei cellobiohydrolase I or II, a Trichoderma reesei endoglucanase I, II or III, and a Trichoderma reesei xylanase II.

D. Vectors

A polynucleotide encoding any of the engineered antibodies disclosed herein can be present in a vector, for example, a phage, plasmid, viral, or retroviral vector. In certain embodiments, the vector can be an expression vector for expressing a subject fusion polypeptide in a filamentous fungal cell.

Vectors for expression of recombinant proteins are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.).

A fusion DNA construct can be constructed using well known techniques as is generally described for example in European Patent Application Publication No. 0 215 594, the disclosure of which is incorporated by reference herein.

Natural or synthetic polynucleotide fragments encoding for the polypeptide of interest (e.g. an immunoglobulin) can be incorporated into heterologous nucleic acid constructs or vectors, capable of introduction into and replication in a host cell (e.g., a filamentous fungal host cell).

Once a DNA construct or more specifically a fusion DNA construct is made it can be incorporated into any number of vectors as is known in the art. While the DNA construct will in some embodiments include a promoter sequence, in other embodiments the vector will include other regulatory sequences functional in the host to be transformed, such as ribosomal binding sites, transcription start and stop sequences, terminator sequences, polyadenylation signals, enhancers and or activators. In some embodiments, a polynucleotide encoding engineered antibodies is inserted into a vector which comprises a promoter, signal sequence and carrier protein at an appropriate restriction endonuclease site by standard procedures. Such procedures and related sub-cloning procedures are deemed to be within the scope of knowledge of those skilled in the art.

Terminator sequences which are recognized by the expression host to terminate transcription can be operably linked to the 3′ end of the fusion DNA construct encoding the engineered antibodies to be expressed. Those of general skill in the art are well aware of various terminator sequences that can be used with host cells, such as, filamentous fungi. Non-limiting examples include the terminator from the Aspergillus nidulans trpC gene (Yelton M. et al., (1984) Proc. Natl. Acad. Sci. USA 81: 1470-1474) or the terminator from the Aspergillus niger glucoamylase genes (Nunberg et al. (1984) Mol. Cell. Biol. 4: 2306-2353) or the terminator from the Trichoderma reesei cellobiohydrolase I gene.

Polyadenylation sequences are DNA sequences which when transcribed are recognized by the expression host to add polyadenosine residues to transcribed mRNA. Examples include polyadenylation sequences from A. nidulans trpC gene (Yelton et al (1984) Proc. Natl. Acad. Sci. USA 81; 1470-1474); from A. niger glucoamylase gene (Nunberg et al. (1984) Mol. Cell. Biol. 4:2306-2315); the A. oryzae or A. niger alpha amylase gene and the Rhizomucor miehei carboxyl protease gene.

In further embodiments, the fusion DNA construct or the vector comprising the fusion DNA construct will contain a selectable marker gene to allow the selection of transformed host cells. Selection marker genes are well known in the art and will vary with the host cell used. Examples of selectable markers include but are not limited to ones that confer antimicrobial resistance (e.g. hygromycin, bleomycin, chloroamphenicol and phleomycin). Genes that confer metabolic advantage, such as nutritional selective markers can also find use. Some of these markers include amdS. Also, sequences encoding genes which complement an auxotrophic defect can be used as selection markers (e.g. pyr4 complementation of a pyr4 deficient A. nidulans, A. awamori or Trichoderma reesei and argB complementation of an argB deficient strain). Reference is made to Kelley et al., (1985) EMBO J. 4: 475-479; Penttila et al., (1987) Gene 61:155-164 and Kinghorn et al (1992) Applied Molecular Genetics of Filamentous Fungi, Blackie Academic and Professional, Chapman and Hall, London, the disclosure of each of which are incorporated by reference herein.

E. Host Cells

The expression cassette or vector can be introduced into a suitable expression host cell, which then expresses the corresponding polynucleotide encoding an engineered antibody.

Suitable host cells include cells of any microorganism (e.g., cells of a bacterium, a protist, an alga, a fungus (e.g., a yeast or filamentous fungus), or other microbe), and can be cells of a bacterium, a yeast, or a filamentous fungus. Fungal expression hosts can be, for example, yeasts, which can also serve as ethanologens. Also suited are mammalian expression hosts such as mouse (e.g., NS0), Chinese Hamster Ovary (CHO) or Baby Hamster Kidney (BHK) cell lines. Other eukaryotic hosts such as insect cells or viral expression systems (e.g., bacteriophages such as M13, T7 phage or Lambda, or viruses such as Baculovirus) are also suitable for producing the polypeptide.

Suitable host cells of the bacterial genera include, but are not limited to, cells of Escherichia, Proteus, Bacillus, Ralstonia, Lactobacillus, Lactococcus, Pseudomonas, Staphylococcus, and Streptomyces. Suitable cells of bacterial species include, but are not limited to, cells of Escherichia coli, Bacillus subtilis, Bacillus licheniformis, Bacillus megaterium, Lactobacillus brevis, Pseudomonas aeruginosa, Pseudomonas fluorescens, Pseudomonas stutzerei, Staphylococcus carnosus, Lactococcus lactis, Ralstonia eutropha, Proteus mirabilis, and Streptomyces lividans.

Suitable host cells of the genera of yeast include, but are not limited to, cells of Saccharomyces, Schizosaccharomyces, Candida, Hansenula, Pichia, Kluyveromyces, Yarrowia and Phaffia. Suitable cells of yeast species include, but are not limited to, cells of Saccharomyces cerevisiae, Schizosaccharomyces pombe, Candida albicans, Hansenula polymorpha, Yarrowia hpolytica, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces jlavus, Thielavia terrestris, Trametes villosa, Trametes versicolor, Trichoderma harzianum, Trichoderma koningii, Trichoderma longibrachiatum, Trichoderma reesei, and Trichoderma viride.

Promoters and/or signal sequences associated with secreted proteins in a particular host of interest are candidates for use in the heterologous production and secretion of engineered antibodies in that host or in other hosts. As a non-limiting example, in filamentous fungal systems, the promoters that drive the genes for cellobiohydrolase I (cbh1), glucoamylase A (glaA), TAKA-amylase (amyA), xylanase (ex1A), the gpdA-promoter cbh1, cbhll, endoglucanase genes eg1-eg5, Cel61B, Cel74A, gpd promoter, Pgk1, pki1, EF-1alpha, tef1, cDNA1 and hex1 are suitable and can be derived from a number of different organisms (e.g., A. niger, T. reesei, A. oryzae, A. awamori, A. nidulans).

In some embodiments, the polynucleotide encoding an engineered antibody is recombinantly associated with a polynucleotide encoding a suitable homologous or heterologous signal sequence that leads to secretion of the recombinant polypeptide into the extracellular (or periplasmic) space, thereby allowing direct detection in the cell supernatant (or periplasmic space or lysate). Suitable signal sequences for Escherichia coli, other gram-negative bacteria and other organisms known in the art include those that drive expression of the HlyA, DsbA, Pbp, PhoA, PelB, OmpA, OmpT or M13 phage Gill genes. For Bacillus subtilis, Gram-positive organisms and other organisms known in the art, suitable signal sequences further include those that drive expression of the AprE, NprB, Mpr, AmyA, AmyE, Blac, SacB, and for S. cerevisiae or other yeast, including the killer toxin, Bar1, Suc2, Mating factor alpha, Inu1A or Ggp1p signal sequence. Signal sequences can be cleaved by a number of signal peptidases, thus removing them from the rest of the expressed protein.

In some embodiments, the engineered antibodies are expressed alone or as a fusion with additional peptides, tags or proteins located at the N- or C-terminus (e.g., 6× His, HA or FLAG tags). Suitable fusions include tags, peptides or proteins that facilitate affinity purification or detection (e.g., 6× His, HA, chitin binding protein, thioredoxin or FLAG tags), as well as those that facilitate expression, secretion or processing of the target beta-glucosidases. In addition to KEX2, further suitable processing sites include enterokinase, STE13, or other protease cleavage sites known in the art for cleavage in vivo or in vitro.

Polynucleotides encoding engineered antibodies can be introduced into expression host cells by a number of transformation methods including, but not limited to, electroporation, lipid-assisted transformation or transfection (“lipofection”), chemically mediated transfection (e.g., CaCl and/or CaP), lithium acetate-mediated transformation (e.g., of host-cell protoplasts), biolistic “gene gun” transformation, PEG-mediated transformation (e.g., of host-cell protoplasts), protoplast fusion (e.g., using bacterial or eukaryotic protoplasts), liposome-mediated transformation, Agrobacterium tumefaciens, adenovirus or other viral or phage transformation or transduction.

F. Pharmaceutical Compositions

Provided herein are pharmaceutical compositions containing an engineered antibody or antigen-binding fragment thereof provided herein. The pharmaceutical composition can be used for therapeutic, prophylactic, and/or diagnostic applications. The engineered antibodies or antigen-binding fragments thereof provided herein can be formulated with a pharmaceutical acceptable carrier or diluent. Generally, such pharmaceutical compositions utilize components which will not significantly impair the biological properties of the antibody or antigen-binding fragment thereof, such as the binding of to its specific epitope (e.g. binding to an epitope on a target protein). Each component is pharmaceutically and physiologically acceptable in the sense of being compatible with the other ingredients and not injurious to the patient. The formulations can conveniently be presented in unit dosage form and can be prepared by methods well known in the art of pharmacy, including but not limited to, tablets, pills, powders, liquid solutions or suspensions (e.g., including injectable, ingestible and topical formulations (e.g., eye drops, gels or ointments), aerosols (e.g., nasal sprays), liposomes, suppositories, injectable and infusible solution and sustained release forms. See, e.g., Gilman, et al. (eds. 1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds. 1993) Pharmaceutical Dosage Forms: Parenteral Medications, Dekker, NY; Lieberman, et al. (eds. 1990) Pharmaceutical Dosage Forms: Tablets, Dekker, NY; and Lieberman, et al. (eds. 1990) Pharmaceutical Dosage Forms: Disperse Systems, Dekker, NY. When administered systematically, the therapeutic composition is sterile, pyrogen-free, generally free of particulate matter, and in a parenterally acceptable solution having due regard for pH, isotonicity, and stability. These conditions are known to those skilled in the art. Methods for preparing parenterally administrable compositions are well known or will be apparent to those skilled in the art and are described in more detail in, e.g., “Remington: The Science and Practice of Pharmacy (Formerly Remington's Pharmaceutical Sciences),” 19th ed., Mack Publishing Company, Easton, Pa. (1995).

Pharmaceutical compositions provided herein can be in various forms, e.g., in solid, semi-solid, liquid, powder, aqueous, or lyophilized form. Examples of suitable pharmaceutical carriers are known in the art and include but are not limited to water, buffering agents, saline solutions, phosphate buffered saline solutions, various types of wetting agents, sterile solutions, alcohols, gum arabic, vegetable oils, benzyl alcohols, gelatin, glycerin, carbohydrates such as lactose, sucrose, amylose or starch, magnesium stearate, talc, silicic acid, viscous paraffin, perfume oil, fatty acid monoglycerides and diglycerides, pentaerythritol fatty acid esters, hydroxy methylcellulose, powders, among others. Pharmaceutical compositions provided herein can contain other additives including, for example, antioxidants, preservatives, antimicrobial agents, analgesic agents, binders, disintegrants, coloring, diluents, excipients, extenders, glidants, solubilizers, stabilizers, tonicity agents, vehicles, viscosity agents, flavoring agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as crystalline cellulose, microcrystalline cellulose, citric acid, dextrin, dextrose, liquid glucose, lactic acid, lactose, magnesium chloride, potassium metaphosphate, starch, among others (see, generally, Alfonso R. Gennaro (2000) Remington: The Science and Practice of Pharmacy, 20th Edition. Baltimore, Md.: Lippincott Williams & Wilkins). Such carriers and/or additives can be formulated by conventional methods and can be administered to the subject at a suitable dose. Stabilizing agents such as lipids, nuclease inhibitors, polymers, and chelating agents can preserve the compositions from degradation within the body.

Pharmaceutical compositions suitable for use include compositions wherein one or more engineered antibodies are contained in an amount effective to achieve their intended purpose. Determination of a therapeutically effective amount is well within the capability of those skilled in the art. Therapeutically effective dosages can be determined by using in vitro and in vivo methods as described herein. Accordingly, an engineered antibody or antigen-binding fragment thereof provided herein, when in a pharmaceutical preparation, can be present in unit dose forms for administration.

An engineered antibody or antigen-binding fragment thereof provided herein can be lyophilized for storage and reconstituted in a suitable carrier prior to use. This technique has been shown to be effective with conventional immunoglobulins and protein preparations and art-known lyophilization and reconstitution techniques can be employed.

An engineered antibody or antigen-binding fragment thereof provided herein can be provided as a controlled release or sustained release composition. Polymeric materials are known in the art for the formulation of pills and capsules which can achieve controlled or sustained release of the antibodies or antigen-binding fragments thereof provided herein (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas (1983) J., Macromol. Sci. Rev. Macromol. Chem. 23:61; see also Levy et al. (1985) Science 228:190; During et al. (1989) Ann. Neurol. 25:351; Howard et al. (1989) J. Neurosurg. 7 1:105; U.S. Pat. Nos. 5,679,377, 5,916,597, 5,912,015, 5,989,463, 5,128,326; PCT Publication Nos. WO 99/15154 and WO 99/20253). Examples of polymers used in sustained release formulations include, but are not limited to, poly(-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. Generally, the polymer used in a sustained release formulation is inert, free of leachable impurities, stable on storage, sterile, and biodegradable. Any technique known in the art for the production of sustained release formulation can be used to produce a sustained release formulation containing one or more engineered antibodies or antigen-binding fragments provided herein.

### III. Methods

A. Methods of Producing Engineered Antibodies

The engineered antibodies disclosed herein (i.e., antibodies incorporating a modified hinge as described supra) can be produced by any method known in the art for the synthesis of antibodies, in particular, by chemical synthesis or by recombinant expression techniques.

Monoclonal antibodies can be prepared using a wide variety of techniques known in the art including the use of hybridoma, recombinant, and phage display technolog or a combination thereof. For example, monoclonal antibodies can he produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with an antigen or immunogenic fragment thereof and once an immune response is detected, e.g., antibodies specific for the administered antigen are detected in the mouse serum, the mouse spleen is harvested and splenocytes isolated. The splenocytes are then fused by well-known techniques to any suitable myeloma cells, for example cells from cell line SP20 available from the ATCC. Additionally, a RIMMS (repetitive immunization, multiple sites) technique can be used to immunize an animal (Kilpatrick et of, 1997, Hybridoma 16:381-9). Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody wherein, the hybridoma may be generated by fusing splenocytes isolated from a mouse immunized with an antigen or immunogenic fragments thereof, with myeloma cells and then screening the hybridomas resulting from the fusion for hybridoma clones that secrete an antibody able to bind the administered antigen.

The engineered antibodies disclosed herein can additionally contain novel amino acid residues in their hinge regions. Engineered antibodies can be generated by numerous methods well known to one skilled in the art. Non-limiting examples include, isolating antibody coding regions (e.g., from hybridoma) and introducing one or more hinge modifications of the invention into the isolated antibody coding region. Alternatively, the variable regions may be subcloned into a vector encoding comprising a modified hinge region (such as any of these disclosed herein). Additional methods and details are provided infra.

Antibody fragments that recognize specific an antigen can be generated by any technique known to those of skill in the art. For example, Fab and F(ab′)2 fragments of the invention can be produced by proteoly tic cleavage of immunoglobulin molecules, using enzymes such as papain (to produce Fab fragments) or pepsin (to produce F(ab′)2 fragments). F(ab′)2 fragments contain the variable region, the light chain constant region and the domain of the heavy chain. Further, the engineered antibodies disclosed herein can also be generated using various phage display methods known in the art.

In phage display methods, functional antibody domains are displayed on the surface of phage particles that carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an Antigen epitope of interest can he selected or identified with antigen, e.g., using labeled antigen or antigen bound or captured to a solid surface or bead. Examples of phage display methods that can be used to make the engineered antibodies disclosed herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Publication Nos. WO 90/02809, WO 91/10737, WO 92/01047, WO 92/18619, WO 93/11236, WO 95/15982, WO 95/20401, and W097/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can he isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12(6): 864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can he cloned into vectors expressing a VH constant region, e.g., the human gamma constant, and the PCR amplified VL domains can be cloned into vectors expressing a VL constant region, e.g., human kappa or lamba constant regions. It is contemplated that the constant region comprises a modified hinge (such as any of the modified hinges disclosed herein). In certain embodiments, the vectors for expressing the VH or VL domains comprise a promoter, a secretion signal, a cloning site for both the variable and constant domains, as well as a selection marker such as neomycin. The VH and VL domains may also be cloned into one vector expressing the desired constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. Methods for producing chimeric antibodies are known in the art. See e.g., Morrison, 1985, Science 229:1202; Oi of al., 1986, BioTechniques 4:214; Gillies et al., 1989, J. Immunol. Methods 125:191-202; and U.S. Pat. Nos. 5,807,715, 4,816,567, 4,816,397, and 6,311,415.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and PCT Publication Nos. WO 98/46645, WO 98/50433, WO 98/24893, W098/16654, WO 96/34096, WO 96/33735, and WO 91/10741.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)2, Fabc, Fv) in which all or substantially all of the CDR regions correspond to those of a non-human immunoglobulin (i.e., donor antibody) and all or substantially all of the framework regions are those of a human immunoglobulin consensus sequence. In a specific embodiment, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can he selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG.sub.1. Where such cytotoxic activity is not desirable, the constant domain may be of the IgG.sub.2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, or greater than 95%. Humanized antibody can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5): 489-498; Studnicka et al., 1994, Protein Engineering 7(6): 805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119-25 (2002), Caldas et al., Protein Eng. 13(5): 353 -60 (2000). Morea et al., Methods 20(3): 267-79 (2000), Baca et al., J. Biol. Chem. 272(16): 10678-84 (1997), Roguska et al., Protein Eng. 9(10): 895-904 (1996), Couto et al., Cancer Res. 55 (23 Supp): 59735 -5977s (1995), Couto et al., Cancer Res. 55(8): 1717-22 (1995), Sandhu J S, Gene 150(2): 409-10 (1994), and Pedersen et al., J. Mol. Biol. 235(3): 959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen or immunogenic fragments thereof. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology fur producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos, 5,413,923, 5,625,126, 5,633,425, 5,569,825, 5,661,016, 5,545,806, 5,814,318, and 5,939,598.

Further, the engineered antibodies disclosed herein can, in turn, be utilized to generate anti-idiotype antibodies that “mimic” a receptor using techniques well known to those skilled in the art. (See, e.g., Greenspan & Bona, 1989, FASEB J 7(5): 437-444; and Nissinoff, 1991, J. Immunol. 147(8): 2429-2438). For example, antibodies of the invention which bind to and competitively inhibit the binding of a receptor (as determined by assays well known in the art and disclosed infra) to its ligands can be used to generate anti-idiotypes that “mimic” the ligand and, as a consequence, bind to and neutralize the receptor and/or its ligands. Such neutralizing anti-idiotypes or Fab fragments of such anti-idiotypes can be used in therapeutic regimens to neutralize a ligand and/or its receptor. Methods employing the use of polynucleotides comprising a nucleotide sequence encoding an engineered antibody or a fragment thereof are provided herein.

In one embodiment, the nucleotide sequence encoding an antibody that specifically binds an antigen is obtained and used to generate the engineered antibodies disclosed herein. The nucleotide sequence can be obtained from sequencing hybridoma clone DNA. If a clone containing a nucleic acid encoding a particular antibody or an epitope-binding fragment thereof is not available, but the sequence of the antibody molecule or epitope-binding fragment thereof is known, a nucleic acid encoding the immunoglobulin may be chemically synthesized or obtained from a suitable source (e.g., an antibody cDNA library, or a cDNA library generated from, or nucleic acid, preferably poly A+RNA., isolated from any tissue or cells expressing the antibody, such as hybridoma cells selected to express an antibody) by PCR amplification using synthetic primers that hybridize to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed metagenesis, PCR, etc. (see, for example, the techniques described in Current Protocols in Molecular Biology, F. M. Ausubel et of, ed., John Wiley & Sons (Chichester, England, 1998), Molecular Cloning: A Laboratory Manual, 3nd Edition, J. Sambrook et al., ed., Cold Spring Harbor Laboratory Press (Cold Spring Harbor, N.Y., 2001); Antibodies: A Laboratory Manual, E. Harlow and D. Lane, ed., Cold Spring Harbor Laboratory Press (Cold Spring Harbor, N.Y., 1988); and Using Antibodies: A Laboratory Manual, E. Harlow and D. Lane, ed., Cold Spring Harbor Laboratory (Cold Spring Harbor, N.Y., 1999)), to generate antibodies having a different amino acid sequence by, for example, introducing deletions, and/or insertions into desired regions of the antibodies.

In a specific embodiment, one or more of the CDRs is inserted within framework regions using routine recombinant DNA techniques. The framework regions may be naturally occurring or consensus framework regions, including, but not limited to, human framework regions (see, e.g., Chothia et al., 1998, J. Mol. Biol. 278: 457-479 for a listing of human framework regions). It is contemplated that the polynucleotide generated by the combination of the framework regions and CDRs encodes an antibody that specifically binds to an Antigen. In one embodiment, as discussed supra, one or more amino acid substitutions may be made within the framework regions, and, in certain embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

The hinge of antibodies identified from such screening methods can be modified as described supra to generate an antibody incorporating a modified hinge, such as any of those disclosed above. It is further contemplated that the engineered antibodies disclosed herein are useful for the prevention, management and treatment of a disease, disorder, infection, including but not limited to inflammatory diseases, autoimmune diseases, bone metabolism related disorders, angiogenic related disorders, infection, and cancer. Such antibodies can be used in the methods and compositions disclosed herein.

B. Recombinant Expression

Recombinant expression of any of the engineered antibodies disclosed herein as well as derivatives, analogs or fragments thereof. (e.g., an antibody or fusion protein of the invention), requires construction of an expression vector containing a polynucleotide that encodes the engineered antibody. Once a polynucleotide encoding an engineered antibody has been obtained, the vector for the production of the engineered antibody can be produced by recombinant DNA technology using techniques well known in the art. Thus, methods for preparing a protein by expressing a polynucleotide containing engineered antibody-encoding nucleotide sequence are described herein. Methods that are well known to those skilled in the art can be used to construct expression vectors containing engineered antibody coding sequences and appropriate transcriptional and translational control signals. These methods include, for example, in vitro recombinant DNA techniques, synthetic techniques, and in vivo genetic recombination.

The expression vector is transferred to a host cell by conventional techniques and the transfected cells are then cultured by conventional techniques to produce an engineered antibody (such as any of those disclosed herein). In specific embodiments for the expression of engineered antibodies comprising double-chained antibodies, vectors encoding both the heavy and light chains may be co-expressed in the host cell for expression of the entire immunoglobulin molecule.

C. Purification of Antibodies

Methods for purification of polypeptides, including the engineered antibodies or antigen-binding fragments thereof provided herein, from host cells will depend on the chosen host cells and expression systems. For secreted molecules, proteins generally are purified from the culture media after removing the cells. For intracellular expression, cells can be lysed and the proteins purified from the extract. In one example, polypeptides are isolated from the host cells by centrifugation and cell lysis (e.g. by repeated freeze-thaw in a dry ice/ethanol bath), followed by centrifugation and retention of the supernatant containing the polypeptides. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary further the proteins can be extracted and further purified using standard methods in the art.

Proteins, such as the engineered antibodies or antigen-binding fragments thereof provided herein, can be purified, for example, from lysed cell extracts, using standard protein purification techniques known in the art including but not limited to, SDS-PAGE, size fraction and size exclusion chromatography, ammonium sulfate precipitation and ionic exchange chromatography, such as anion exchange. Affinity purification techniques also can be utilized to improve the efficiency and purity of the preparations. For example, antibodies, receptors and other molecules that bind proteases can be used in affinity purification. Expression constructs also can be engineered to add an affinity tag to a protein such as a myc epitope, GST fusion or His6 and affinity purified with myc antibody, glutathione resin and Ni-resin, respectively. Purity can be assessed by any method known in the art including gel electrophoresis and staining and spectrophotometric techniques.

Typically, antibodies and portions thereof are purified by any procedure known to one of skill in the art. The antibodies can be purified to substantial purity using standard protein purification techniques known in the art including but not limited to, SDS-PAGE, size fraction and size exclusion chromatography, ammonium sulfate precipitation, chelate chromatography, ionic exchange chromatography or column chromatography. For example, antibodies can be purified by column chromatography. Exemplary of a method to purify antibodies is by using column chromatography, wherein a solid support column material is linked to Protein G, a cell surface-associated protein from Streptococcus, that binds immunoglobulins with high affinity. The antibodies can be purified to 60%, 70%, 80% purity and typically at least 90%, 91%, 92%, 93%, 94%, 95%, 96%, 97%, 98% or 99% purity. Purity can be assessed by standard methods such as by SDS-PAGE and coomassie staining.

The isolated polypeptides then can be analyzed, for example, by separation on a gel (e.g. SDS-Page gel), size fractionation (e.g. separation on a Sephacryl™ S-200 HiPrep™ 16×60 size exclusion column (Amersham from GE Healthcare Life Sciences, Piscataway, N.J.). Isolated polypeptides also can be analyzed in binding assays, typically binding assays using a binding partner bound to a solid support, for example, to a plate (e.g. ELISA-based binding assays) or a bead, to determine their ability to bind desired binding partners. The binding assays described in the sections below, which are used to assess binding of precipitated phage displaying the polypeptides, also can be used to assess polypeptides isolated directly from host cell lysates. For example, binding assays can be carried out to determine whether antibody polypeptides bind to one or more antigens, for example, by coating the antigen on a solid support, such as a well of an assay plate and incubating the isolated polypeptides on the solid support, followed by washing and detection with secondary reagents, e.g. enzyme-labeled antibodies and substrates.

D. Administration of Pharmaceutical Compositions

Also provided herein are methods and pharmaceutical compositions comprising any of the engineered antibodies disclosed herein (such as any antibody comprising the modified hinge regions disclosed herein). Further provided herein are methods of treatment, prophylaxis, and amelioration of one or more symptoms associated with a disease, disorder or infection by administering to a subject an effective amount of at least one engineered antibody disclosed herein, or a pharmaceutical composition comprising at least one engineered antibody disclosed herein. In a one aspect, the engineered antibody is substantially purified (i.e., substantially free from substances that limit its effect or produce undesired side-effects.). In a specific embodiment, the subject is an animal, such as a mammal including non-primates (e.g., cows, pigs, chickens or other fowl, horses, cats, dogs, rats etc.) and primates (e.g., monkey such as, a cynomolgous monkey and a human). In a specific embodiment, the subject is a human. In yet another specific embodiment, the antibody of the invention is from the same species as the subject.

The route of administration of the composition depends on the condition to be treated. For example, intravenous injection may be preferred for treatment of a systemic disorder such as a lymphatic cancer or a tumor which has metastasized. The dosage of the compositions to he administered can be determined by the skilled artisan without undue experimentation in conjunction with standard dose-response studies. Relevant circumstances to be considered in making those determinations include the condition or conditions to be treated, the choice of composition to he administered, the age, weight, and response of the individual patient, and the severity of the patient's symptoms. Depending on the condition, the composition can be administered orally, parenterally, intranasally, vaginally, rectally, lingually, sublingually, buccally, intrabuccally and/or transdermally to the patient.

Accordingly, compositions designed for oral, lingual, sublingual, buccal and intrabuccal administration can be made without undue experimentation by means well known in the art, for example, with an inert diluent or with an edible carrier. The composition may be enclosed in gelatin capsules or compressed into tablets. For the purpose of oral therapeutic administration, the pharmaceutical compositions of the present invention may be incorporated with excipients and used in the form of tablets, pellets, troches, capsules, elixirs, suspensions, syrups, wafers, chewing gums, and the like. In further embodiments, the composition can be incorporated into an animal feed.

Tablets, pills, capsules, troches and the like may also contain binders, recipients, disintegrating agent, lubricants, sweetening agents, and/or flavoring agents. Some examples of binders include microcrystalline cellulose, gum tragacanth and gelatin. Non-limiting examples of excipients include starch and lactose. Some examples of disintegrating agents include alginic acid, cornstarch, and the like. Examples of lubricants include magnesium stearate and potassium stearate. An example of a glidant is colloidal silicon dioxide. Some non-limiting examples of sweetening agents include sucrose, saccharin, and the like. Examples of flavoring agents include peppermint, methyl salicylate, orange flavoring, and the like. Materials used in preparing these various compositions should be pharmaceutically pure and non-toxic in the amounts used.

The pharmaceutical compositions disclosed herein can be administered parenterally, such as, for example, by intravenous, intramuscular, intrathecal andlor subcutaneous injection. Parenteral administration can be accomplished by incorporating the compositions disclosed herein into a solution or suspension. Such solutions or suspensions may also include sterile diluents, such as water for injection, saline solution, fixed oils, polyethylene glycols, glycerine, propylene glycol and/or other synthetic solvents. Parenteral formulations may also include antibacterial agents, such as, for example, benzyl alcohol and/or methyl parabens, antioxidants, such as, for example, ascorbic acid and/or sodium bisulfite, and chelating agents, such as EDTA. Buffers, such as acetates, citrates and phosphates, and agents for the adjustment of tonicity, such as sodium chloride and dextrose, may also be added. The parenteral preparation can be enclosed in ampules, disposable syringes and/or multiple dose vials made of glass or plastic. Rectal administration includes administering the composition into the rectum and/or large intestine. This can be accomplished using suppositories and/or enemas. Suppository formulations can be made by methods known in the art. Transdermal administration includes percutaneous absorption of the composition through the skin. Transdermal formulations include patches, ointments, creams, gels, salves, and the like. The engineered antibody-containing compositions disclosed herein can be administered nasally to a patient. As used herein, nasally administering or nasal administration includes administering the compositions to the mucous membranes of the nasal passage and/or nasal cavity of the patient.

The engineered antibody-containing compositions disclosed herein can be used in accordance with the methods of the invention for preventing, treating, or ameliorating one or more symptoms associated with a disease, disorder, or infection. It is contemplated that the pharmaceutical compositions of the invention are sterile and in suitable form for administration to a subject.

In one embodiment, the engineered antibody-containing compositions disclosed herein are pyrogen-free formulations which are substantially free of endotoxins and/or related pyrogenic substances. Endotoxins include toxins that are confined inside a microorganism and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with monoclonal antibodies, it is advantageous to remove even trace amounts of endotoxin. In a specific embodiment, endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

Additionally provided herein are methods for preventing, treating, or ameliorating one or more symptoms associated with a disease, disorder, or infection, said method comprising: (a) administering to a subject in need thereof a dose of a prophylactically or therapeutically effective amount of a composition comprising one or more of the engineered antibodies disclosed herein and (b) administering one or more subsequent doses of said engineered antibodies, to maintain a. plasma concentration of the engineered antibodies at a desirable level (e.g., about 0.1 to about 100 μg/ml), which continuously binds to an antigen. In a specific embodiment, the plasma concentration of the engineered antibodies is maintained at 10 μg/ml, 15 μg/ml, 20 μg/ml, 25 μg/ml, 30 μg/ml, 35 μg/ml, 40 μg/ml, 45 μg/ml or 50 μg/ml. In a specific embodiment, said effective amount of engineered antibodies to be administered is between at least 1 mg/kg and 8 mg/kg per dose. In another specific embodiment, said effective amount of engineered antibodies to be administered is between at least 4 mg/kg and 8 mg/kg per dose. In yet another specific embodiment, said effective amount of engineered antibodies to be administered is between 50 mg and 250 mg per dose. In still another specific embodiment, said effective amount engineered antibodies to be administered is between 100 mg and 200 mg per dose.

Also provided herein are protocols for preventing, treating, or ameliorating one or more symptoms associated with a disease, disorder, or infection which an any of the engineered antibodies disclosed herein is used in combination with a therapy (e.g., prophylactic or therapeutic agent). The engineered antibodies disclosed herein can potentiate and synergize with, enhance the effectiveness of improve the tolerance of, and/or reduce the side effects caused by, other cancer therapies, including current standard and experimental chemotherapies. The combination therapies of the invention have additive potency, an additive therapeutic effect or a synergistic effect. The combination therapies of the invention enable lower dosages of the therapy prophylactic or therapeutic agents) utilized in conjunction with the engineered antibodies disclosed herein for preventing, treating, or ameliorating one or more symptoms associated with a disease, disorder, or infection and/or less frequent administration of such prophylactic or therapeutic agents to a subject with a disease disorder, or infection to improve the quality of life of said subject and/or to achieve a prophylactic or therapeutic effect. Further, the combination therapies of the invention reduce or avoid unwanted or adverse side effects associated with the administration of current single agent therapies and/or existing combination therapies, which in turn improves patient compliance with the treatment protocol. Numerous molecules which can be utilized in combination with the engineered antibodies disclosed herein are well known in the art. See for example, PCT publications WO 02/070007; WO 03/075957 arid U.S. Patent Publication 2005/064514.

E. Assessing Engineered Antibody Properties and Activities

1. Binding Assays

The engineered antibodies or antigen-binding fragments thereof provided herein can be assessed for their ability to bind a selected target and the specificity for such targets by any method known to one of skill in the art. Exemplary assays are provided in the Examples and described herein below. Binding assays can be performed in solution, suspension or on a solid support. For example, target antigens can be immobilized to a solid support (e.g. a carbon or plastic surface, a tissue culture dish or chip) and contacted with antibody or antigen-binding fragment thereof. Unbound antibody or target protein can be washed away and bound complexes can then be detected. Binding assays can be performed under conditions to reduce nonspecific binding, such as by using a high ionic strength buffer (e.g., 0.3-0.4 M NaCl) with nonionic detergent (e.g. 0.1% TRITON X®-100 or TWEEN® 20) and/or blocking proteins (e.g. bovine serum albumin or gelatin). Negative controls also can be included in such assays as a measure of background binding. Binding affinities can be determined using Scatchard analysis (Munson et al., (1980) Anal. Biochem., 107:220), surface plasmon resonance, isothermal calorimetry, or other methods known to one of skill in the art.

Exemplary immunoassays which can be used to analyze immunospecific binding and cross-reactivity include, but are not limited to, competitive and non-competitive assay systems using techniques such as, but not limited to, western blots, radioimmunoassays, ELISA (enzyme linked immunosorbent assay), Meso Scale Discovery (MSD, Gaithersburg, Md.), “sandwich” immunoassays, immunoprecipitation assays, ELISPOT, precipitin reactions, gel diffusion precipitin reactions, immunodiffusion assays, agglutination assays, complement-fixation assays, immunoradiometric assays, fluorescent immunoassays, protein A immunoassays. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Other assay formats include liposome immunoassays (LIA), which use liposomes designed to bind specific molecules (e.g., antibodies) and release encapsulated reagents or markers. The released chemicals are then detected according to standard techniques (see Monroe et al., (1986) Amer. Clin. Prod. Rev. 5:34-41). Exemplary immunoassays not intended by way of limitation are described briefly below.

Immunoprecipitation protocols generally involve lysing a population of cells in a lysis buffer such as RIPA buffer (1% NP-40 or TRITON® X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody or antigen-binding fragment thereof of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody or antigen-binding fragment thereof of interest to immunoprecipitate a particular antigen can be assessed by, e.g., western blot analysis. One of skill in the art is knowledgeable as to the parameters that can be modified to increase the binding of the antibody or antigen-binding fragment thereof to an antigen and decrease the background (e.g., pre-clearing the cell lysate with sepharose beads). For further discussion regarding immunoprecipitation protocols see, e.g., Ausubel et al., eds, 1994, Current Protocols in Molecular Biology, Vol. 1, John Wiley & Sons, Inc., New York at 10.16.1.

Western blot analysis generally involves preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody or antigen-binding fragment thereof (i.e., the antibody or antigen-binding fragment thereof of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art is knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs involve preparing antigen, coating the well of a 96-well microtiter plate with the antigen, adding the antibody or antigen-binding fragment thereof of interest conjugated to a detectable compound such as an enzymatic substrate (e.g., horseradish peroxidase or alkaline phosphatase) to the well and incubating for a period of time, and detecting the presence of the antigen. In ELISAs, the antibody or antigen-binding fragment thereof of interest does not have to be conjugated to a detectable compound; instead, a second antibody (which recognizes the antibody of interest) conjugated to a detectable compound can be added to the well. Further, instead of coating the well with the antigen, the antibody can be coated to the well. In this case, a second antibody conjugated to a detectable compound can be added following the addition of the antigen of interest to the coated well. One of skill in the art is knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody or antigen-binding fragment thereof to an antigen and the off-rate of an antibody-antigen interaction can be determined, for example, by competitive binding assays. One example of a competitive binding assay is a radioimmunoassay comprising the incubation of labeled antigen (e.g., ³H or ¹²⁵I) with the antibody or antigen-binding fragment thereof of interest in the presence of increasing amounts of unlabeled antigen, and the detection of the antibody or antigen-binding fragment thereof bound to the labeled antigen. The affinity of an engineered antibody or antigen-binding fragment thereof provided herein for an antigen and the binding off-rates can be determined from the data by Scatchard plot analysis. Competition with a second antibody can also be determined using radioimmunoassays. In this case, an antigen is incubated with an engineered antibody or antigen-binding fragment thereof provided herein conjugated to a labeled compound (e.g., ³H or ¹²⁵I) in the presence of increasing amounts of an unlabeled second antibody. In some examples, surface plasmon resonance (e.g., BiaCore 2000, Biacore AB, Upsala, Sweden and GE Healthcare Life Sciences; Malmqvist (2000) Biochem. Soc. Trans. 27:335) kinetic analysis can be used to determine the binding on and off rates of antibodies or antigen-binding fragments thereof to an antigen. Surface plasmon resonance kinetic analysis involves analyzing the binding and dissociation of an antigen from chips with immobilized antibodies or fragments thereof on their surface.

2. Binding Specificity

The binding specificity, or epitope, of the engineered antibodies or antigen binding fragments thereof provided herein can be determined by any assay known to one of skill in the art, including, but not limited to surface plasmon resonance assays, competition assays and virus neutralization assays using Monoclonal Antibody-Resistant Mutants (MARMs). The ability of two antibodies to bind to the same epitope can be determined by known assays in the art such as, for example, surface plasmon resonance assays and antibody competition assays. Typically, antibodies that immunospecifically bind to the same epitope can compete for binding to the epitope, which can be measured, for example, by an in vitro binding competition assay (e.g. competition ELISA), using techniques known the art. Typically, a first antibody that immunospecifically binds to the same epitope as a second antibody can compete for binding to the epitope by about or 30%, 35%, 40%, 45%, 50%, 55%, 60%, 65%, 70%, 75%, 80%, 85%, 90%, 95%, 100%, where the percentage competition is measured ability of the second antibody to displace binding of the first antibody to the epitope. In exemplary competition assays, the antigen is incubated in the presence a predetermined limiting dilution of a labeled antibody (e.g., 50-70% saturation concentration), and serial dilutions of an unlabeled competing antibody. Competition is determined by measuring the binding of the labeled antibody to the antigen for any decreases in binding in the presence of the competing antibody. Variations of such assays, including various labeling techniques and detection methods including, for example, radiometric, fluorescent, enzymatic and colorimetric detection, are known in the art.

F. Diagnostic Uses

The engineered antibodies or antigen-binding fragments thereof provided herein can be used in diagnostic assays for the detection, purification, and/or neutralization of pathogens. Exemplary diagnostic assays include in vitro and in vivo detection of pathogens. For example, assays using the engineered antibodies or antigen-binding fragments thereof provided herein for qualitatively and quantitatively measuring levels of pathogen in an isolated biological sample (e.g., sputum) or in vivo are provided.

As described herein, the engineered antibodies or antigen-binding fragments thereof can be conjugated to a detectable moiety for in vitro or in vivo detection. Such antibodies can be employed, for example, to evaluate the localization and/or persistence of the engineered antibody or antigen-binding fragment thereof at an in vivo site, such as, for example, a mucosal site. The engineered antibodies or antigen-binding fragments thereof which are coupled to a detectable moiety can be detected in vivo by any suitable method known in the art. The engineered antibodies or antigen-binding fragments thereof which are coupled to a detectable moiety also can be detected in isolated biological samples, such as tissue or fluid samples obtained from the subject following administration of the antibody or antigen-binding fragment thereof.

G. Routes of Administration

The engineered antibodies or antigen-binding fragments thereof provided herein can be administered to a subject by any method known in the art for the administration of polypeptides, including for example systemic or local administration. The engineered antibodies or antigen-binding fragments thereof can be administered by routes, such as parenteral (e.g., intradermal, intramuscular, intraperitoneal, intravenous, subcutaneous, or intracavity), topical, epidural, or mucosal (e.g. intranasal or oral). The engineered antibodies or antigen-binding fragments thereof can be administered externally to a subject, at the site of the disease for exertion of local or transdermal action. Compositions containing engineered antibodies or antigen-binding fragments thereof can be administered by any convenient route, for example by infusion or bolus injection, by absorption through epithelial or mucocutaneous linings (e.g., oral mucosa, rectal and intestinal mucosa). Compositions containing the engineered antibodies or antigen-binding fragments can be administered together with other biologically active agents. The mode of administration can include topical or other administration of a composition. The engineered antibodies or antigen-binding fragments thereof provided herein can be administered by topical or aerosol routes for delivery directly to target organs, such as the lungs (e.g. by pulmonary aerosol). In some examples, the provided engineered antibodies or antigen-binding fragments thereof can be administered as a controlled release formulation as such as by a pump (see, e.g., Langer (1990) Science 249:1527-1533; Sefton (1987) CRC Crit. Ref. Biomed. Eng. 14:20; Buchwald et al. (1980) Surgery 88:507; and Saudek et al. (1989) N. Engl. J. Med. 321:574) or via the use of various polymers known in the art and described elsewhere herein. In some examples, a controlled or sustained release system can be placed in proximity of the therapeutic target, for examples, the lungs, thus requiring only a fraction of the systemic dose (see, e.g., Goodson, in Medical Applications of Controlled Release, supra, vol. 2, pp. 115-138 (1984)).

In particular examples, the provided antibodies or antigen-binding fragments thereof are administered by pulmonary delivery (see, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903). Exemplary methods of pulmonary delivery are known in the art and include, but are not limited to, aerosol methods, such as inhalers (e.g., pressurized metered dose inhalers (MDI), dry powder inhalers (DPI), nebulizers (e.g., jet or ultrasonic nebulizers) and other single breath liquid systems), intratracheal instillation and insufflation. In some examples, pulmonary delivery can be enhanced by co-administration of or administration of a co-formulation containing the engineered antibodies or antigen-binding fragments thereof provided herein and a permeation enhancer, such as, for example, surfactants, fatty acids, saccharides, chelating agents and enzyme inhibitors, such as protease inhibitors.

Appropriate methods for delivery, such as pulmonary delivery, can be selected by one of skill in the art based on the properties of the dosage amount of the antibody or antigen-binding fragment thereof or the pharmaceutical composition containing the antibody or antigen-binding fragment thereof. Such properties include, but are not limited to, solubility, hygroscopicity, crystallization properties, melting point, density, viscosity, flow, stability and degradation profile.

### IV. Kits

Further provided herein are kits comprising One or more of the engineered antibodies disclosed herein with altered (such as, improved) stability and/or decreased potential for proteolytic cleavage that specifically bind to an antigen conjugated or fused to a detectable agent, therapeutic agent or drug, in one or more containers, for use in monitoring, diagnosis, preventing, treating, or ameliorating one or more symptoms associated with a disease, disorder, or infection.

The kit can, optionally, include instructions. Instructions typically include a tangible expression describing the engineered antibodies or antigen-binding fragments thereof and, optionally, other components included in the kit, and methods for administration, including methods for determining the proper state of the subject, the proper dosage amount, dosing regimens, and the proper administration method for administering the engineered antibodies or antigen-binding fragments thereof. Instructions also can include guidance for monitoring the subject over the duration of the treatment time

Kits also can include a pharmaceutical composition described herein and an item for diagnosis. For example, such kits can include an item for measuring the concentration, amount or activity of the selected engineered antibody or antigen-binding fragment thereof in a subject.

In some examples, the engineered antibody or antigen-binding fragment thereof is provided in a diagnostic kit for the detection of a pathogen in an isolated biological sample (e.g., a fluid sample, such as blood, sputum, lavage, lung intubation sample, saliva, urine or lymph obtained from a subject). In some examples, the diagnostic kit contains a panel of one or more engineered antibodies or antigen-binding fragments thereof and/or one or more control antibodies (i.e. non-pathogenic epitope binding antibodies), where one or more antibodies in the panel is an engineered antibody or antigen-binding fragment provided herein.

Kits provided herein also can include a device for administering the engineered antibodies or antigen-binding fragments thereof to a subject. Any of a variety of devices known in the art for administering medications to a subject can be included in the kits provided herein. Exemplary devices include, but are not limited to, an inhaler (e.g., pressurized metered dose inhaler (MDI), dry powder inhaler (DPI), nebulizer (e.g., jet or ultrasonic nebulizers) and other single breath liquid system), a hypodermic needle, an intravenous needle, a catheter, and a liquid dispenser such as an eyedropper. Typically, the device for administering the engineered antibodies or antigen-binding fragments thereof of the kit will be compatible with the desired method of administration of the engineered antibodies or antigen-binding fragments thereof. For example, an engineered antibody or antigen-binding fragment thereof to be delivered by pulmonary administration can be included in a kit with or contained in an inhaler or a nebulizer.

The invention can be further understood by reference to the following examples, which are provided by way of illustration and are not meant to be limiting.

### EXAMPLES

### Example 1

### Backbone Molecule Vector Construction

Both the heavy and light chains of each antibody candidate were constructed on one telomeric vector. This permitted transforming into just one vector into production hosts rather than having each chain on its own vector or making intermediate strains.

Each antibody backbone needed to have integrated a full wild-type expression cassette of either chain into the vector to allow for the opposite chain variant to be assembled. For example, if a wild-type heavy chain were inserted into the host vector, then it will act as the light chain variant library entry vector, and vice versa. The host vector contains a T. reesei cbhl promoter and terminator regions allowing for a strong inducible expression of a gene of interest, the T. reesei pyr2 selective marker conferring growth of transformants on minimal medium in the absence of uridine, and the amdS selective marker conferring growth of transformants on minimal media using acetamide as the sole nitrogen source. The plasmids are maintained autonomously in fungal cells due to T. reesei derived telomere regions. Usage of replicative plasmids resulted in increased frequencies of transformation and circumvented problems of locus-dependent expression observed with integrative fungal transformation. The expression vector also contains CBH1 core exons 1 and 2 and partially 3, wherein there it is cut off and the sequence of ccdB and chloramphenicol resistance marker is between this and the partial CBH1 terminator. The ccdB gene requires a special strain of E. coli to propagate.

Twist biosciences (San Francisco, CA) generated synthetic DNA of wild type antibody chains and cloned each single chain the preferred expression vector. This expression vector contains the T. reesei cbhI promoter and terminator regions allowing for a strong inducible expression of a gene of interest. The cassette will have the 48mer adaptor and homology on the 5′ end to the host vector, CBH1 promoter, CBH1 core, HC or LC, cbh1 terminator and homology to the host vector at the 3′ end. These cassettes were generated by PCR and Dpnl treated before being purified. Both fragments were combined and seamless assembled (Geneart) and transformation into commercially available Escherichia coli ccdB survival cells (Invitrogen, US). Four to six colonies per variant combination were picked, plasmid purified, and diagnostically cut with restriction enzyme Xmal and analyzed on an agarose gel to assess which clones were correctly assembled. The correctly assembled vectors were then sent to sanger sequencing for the HC or LC to ensure correct sequences. Correct plasmids were propagated in commercially available Escherichia coli ccdB survival cells (Invitrogen, US), purified, and cut with Spel and Sphl and gel purified in preparation for inserting the variant opposite chain. The vector that contained the antibody BiiB HC was cut with AsiSI and SphI instead as SpeI cuts in the HC region.

The variant chain fragments are generated by Twist biosciences (San Francisco, Calif.). The variant chain fragment will fill in the cbhl core missing from the plasmid with homology at the 5′ end, HC or LC itself, CBH1 terminator missing portion with homology at the 3′ end. The variant fragments were PCR amplified and purified. This and the entry vectors were combined and seamless assembled (Geneart) and transformation into commercially available Escherichia coli TOP10 cells (Invitrogen, US). Eight colonies per variant combination were picked, plasmid purified, and diagnostically cut with restriction enzyme Notl and analyzed on the ZAG fragment analyzer (Advanced Analytical) to assess which clones were correctly assembled. The correctly assembled vectors were then sent to sanger sequencing for the HC or LC to ensure correct sequences. Correct plasmids were propagated in commercially available Escherichia coli TOP10 cells (Invitrogen, US), purified, arrayed individually in 96 well MTPs and used for fungal transformation.

### Example 2

### Transformation, Culturing, and Purification of Antibody Backbone Variants

The host T. reesei strain used for transformation was deleted for major cellulases and xylanases. The strain was transformed using a standard PEG-protoplast transformation method in high throughput fashion in 24 well MTP formant using Biomek robots (Beckman Coulter, USA). Transformation mixtures containing approximately 1 mg of DNA and 5×10⁶ protoplasts in a total volume of 50 ml were treated with 200 μl of 25% PEG solution, diluted with 1 volumes of 1.2M sorbitol/10 mM Tris, pH7.5/10 mM CaCl₂ solution, rearranged robotically into 24 well MTPs and poured in 1 ml of 3% low melting agarose containing 1M sorbitol and 0.6 g/L acetamide as the sole nitrogen source in minimal medium to maintain double selection of pyr2 and amdS. After sufficient growth transformants from each well were pooled together and plated on fresh 24 well agar plates with 0.6 g/L acetamide as sole nitrogen source in minimal medium to maintain double selection. Once sporulated, spores were harvested and used for inoculation of liquid cultures.

To generate sufficiently high antibody titers, 10⁵-10⁶ T. reesei spores were inoculated in customer-made 24 well MTPs composed of the Sylgard 170 elastomer (from Dow Corning, USA) premixed with lactose which was slowly released in the medium during fermentation to ensure continuous production. Cultures were grown in 1 ml of medium containing: 16 g/L glucose, 9 g/L casamino acids, 10 g/L (NH₄)2SO₄, 4.5 g/L KH₂PO₄, 1 g/L MgSO₄*7H₂O, 1 g/L CaCl₂*2H₂O, 33 g/L PIPPS buffer [pH 5.5], 0.25% T. reesei trace elements (100%: 175 g/L citric acid (anhydrous), 200 g/L FeSO₄*7H₂O, 16 g/L ZnSO₄*7H₂O, 3.2 g/L CuSO₄*5H₂O, 1.4 g/L MnSO₄*H₂O, 0.8 g/L H₃BO₃).

Plates were incubated in an Infors shaker with a 50 mm throw at 200 rpm and 28C with 80% humidity. After 5-6 days of growth cultures were reformatted back to 96 well deep well MTPs using Biomek robots and filtered using 96-well microtiter filter plates 3.0 um Glass Fiber/0.2 urn Supor membrane, Pall, Port Washington N.Y.). The plates were frozen in Axygen half-deep well plates (P-DW-11-C, Corning, Tewksbury Mass.).

Plates were moved from the freezer to the cold room to allow the samples to gradually thaw overnight at 4° C. Before purification, grown WT samples were removed from the plates and these samples were pooled. One mL per well of pooled WT, pooled low binding control, pooled high binding control, and pooled vector only (vector expressing CBH1 in same strain) samples were added to designated wells. The library plates were grown in duplicate and these controls were added to both plates. The plates gently shook for 2 minutes to homogenize the fluid in the wells followed by centrifugation for 1 minute to pellet any precipitate.

The centrifuged plates were then moved to a robot to remove 20 μL of the crude material for Octet Protein A quantitation. The 20 μL was added to 80 μL of 1× PBS in a 384-well plate (Greiner Bio-One 781209). Four library plates went into one 384-well plate and there was a separate 384-well plate for the duplicate growth of the four plates (plates Xa and Xb).

After samples were removed for the Octet quantitation, the plates were then purified. The robot handled four library plates at a time. The robot added 50 μL of 1 M KPi pH 7 to pH up the supernatant to improve the antibody binding to the Protein A resin. The robot then transferred the crude material (max 880 μL per well) from the four plates to 2 mL filter plates (Pall 8275) filled previously with 220 μL of Protein A resin in PBS. These filter plates then shook for 5 minutes on a shaker. The plates were then filtered by centrifugation at 1000 g for 2 minutes, and the flow through was collected in the empty harvest plate that the samples were transferred from. This material was stored until after quantitation. The filter plates were returned to the robot deck and the duplicate growth plates were added to the same filter plates. These plates were incubated and centrifuged as before. The resin was then washed with 880 μL of PBS buffer. The plates shook for 1 minute and then centrifuged at 1000 g for 2 minutes. The flow through was discarded, and the plates were returned to the robot for the second PBS washing. After the second washing, the plates were moved to a robot running the elution program.

The elution program handled four plates at a time. It added 11 μL of neutralization buffer (1 M Tris pH 9) to a clean half-deep well plate that the samples would be eluted into. The program then added 440 μL of elution buffer (100 mM glycine pH 2.7) to the filter plates. The plates then shook for 1 minute at setting 7 and then were filtered by centrifugation (1000 g for 2 minutes) into the freshly prepped recovery plates. After centrifugation, the sample plates shook for 1 minute to ensure proper mixing of the neutralization buffer.

FRET Quantitation Assay and Normalization: Protein A (Thermo Fisher Scientific 77674) was labeled with Alexa Fluor 546 NHS ester (Thermo Fisher Scientific A20102). Protein L (Thermo Fisher Scientific 77680) was labeled with Alexa Fluor 488 NHS ester (Thermo Fisher Scientific A20100). The labeled Protein A and Protein L were diluted with 107 mM KPi pH 7 and at a ratio that produced a FRET signal for the standard curve with the proper dynamic range. The standard curve was commercial Synagis from AbbVie. In a Corning 3605 plate, 40 μL of the Protein A and L solution was mixed with 10 μL of the purified antibody sample. The FRET signal on the plate was read (ex: 485 nm em: 590 nm cutoff: 590 nm), and the concentration of the unknowns was determined from the Synagis standard curve. The samples were run in duplicate.

After analyzing the data, the plates were normalized to 120 ppm. The dilution buffer was Tris-Gly buffer that was at the same pH and concentration is in the purified samples. For the wells that were less than 120 ppm, they were not diluted and were used as is.

### Example 3

### Assaying Backbone Variants for Thermostability and Protease Resistance

Octet Protein L Assay: This assay determines the integrity of the expressed and purified molecule by comparing how well the unknowns bind to Protein L relative to Protein A. The purified and protein normalized plates were diluted 5-fold into PBS buffer in a 384-well plate (Greiner 781209), with four 96-well plates being combined into a single 384-well plate. The 384-well plates were analyzed by the Fortebio Octet HTX. The Octet tips were loaded with Protein A Biosensors (Part #18-5010). A second 384-well plate containing the following solutions was also loaded into the Octet, with solution in a different quadrant of the plate.

The assay utilized neutralization buffer (PBSA—PBS Buffer with 0.01% BSA); Regeneration Buffer (10 mM Glycine pH 1.5); buffer (PBSA); and a detection reagent (100 ug/ml Protein-L (Pierce REF21189) in PBSA).

The Octet instrument was set up as follows: sample load (96 tip array positions into 1st set of samples. 180 seconds, 400 rpm); buffer (sample loaded sensors position into PBSA to wash off excess sample. 20 seconds, 1000 rpm); detection (sensors move to wells containing Protein-L, 60 seconds, 400 rpm); regeneration/neutralization (3 cycles of each. 5 seconds, 1000 rpm); repeat for the rest of the 96-well plates that make up the 384-well plate.

The Octet data was processed as follows to calculate a PI (Performance Index), which is needed to compare samples across different plates. The equilibrium binding rate for the Protein L association step was divided by the equilibrium binding for the load step to the Protein A sensor. This ratio for each variant was then normalized by dividing the variant ratio by the ratio for the WT on each plate. This normalized value is the PI. A sample that has a PI greater than 1 has more intact FAB region than WT, and a sample with a PI less than 1 has a less intact FAB region than WT.

FAB Stability Assay: To test the stability of the F(ab′)₂, the hinge was cleaved with a commercial protease that digests IgG at a specific site below the hinge region, generating a homogenous pool of F(ab′)₂ and Fc/2 fragments. This protease is FabRICATOR (Genovis A0-FR1-050), and the cleavage reaction was conducted as described by the manufacturer. The antibody variants used in the cleavage reaction and in the stability assay were not protein normalized and were not further purified.

The samples were diluted between 4-fold and 10-fold in PBS to optimize the Octet unstress signal for each backbone library. This dilution was in a 96-well PCR plate with a final volume of 220 μL, and 100 μL of this diluted material was transferred to a 384-well Octet plate (Greiner 781209) for the unstress measurement. The remaining solution in the PCR plate was stressed in a thermocycler for 10 minutes at an elevated temperature. After the temperature stress, 100 μL of stressed sample was transferred to a 394-well Octet plate and read. The elevated temperature was set to yield a % residual signal (%RS) of 20˜30% for the WT for each backbone. The %RS is defined as the equilibrium binding rate of the stressed sample divided by the equilibrium binding rate of the unstressed sample and then multiplied by 100. To compare mutations across different plates and backbones, the %RS was converted to a Performance Index (PI) by dividing the %RS of a variant by the %RS of the WT-backbone on the same plate. There was a slight concentration dependence for the stability data. The more concentrated samples (those having a higher unstress binding rate) appeared less stable. This was evident by plotting PI versus unstressed binding rate, which yielded a negative correlation that was linear. This slope was determined for each backbone and used to correct the PIs. After the correction, the plot of corrected PIs versus unstressed binding rate yielded a slope of zero. The Octet probes used in this assay were Anti-Human Fab-CH1 (FABG2, Part #18-5125), and the instrument was the Fortebio Octet HTX.

Protein thermal shift (“Tm”) assay using SYPRO® Orange and PCR thermocycler: Unfolding of purified antibody polypeptide (including wild type and variants) was measured as follows. To 10 μl of purified and protein normalized antibody at 120ppm in the 384 well plate (Roche Diagnostics, Indianapolis, Ind.), 5 μl of 100 mM Cellobiose in 200 mM NaAc pH5 was added and mixed, then 5 μl of 250-fold diluted sypro orange (Thermo Fisher Scientific Fisher, Grand Island, N.Y.) was added and mixed. The plate was sealed and placed in the Roche Lightcycler 480. A program with 5 minutes at 37° C., a gradient from 37° C. to 97° C. with a heating ramp rate of 0.02° C./sec and 38 acquisitions/° C. was run, the relative fluorescence change at EX 472 nm/EM 570 nm was recorded. The data was exported and was processed using R scripts. The first derivative of the fluorescence change vs. temperature is calculated, the transition temperatures corresponding to Fc, and Fab unfolding were reported.

Thermal Stability: In a 96-well PCR plate, 30 μL of 150 mM Na Acetate pH 5 was mixed with 70 μL of normalized antibody sample. For the unstressed FRET read, 20 μL of this antibody mixture was added to 30 μL of the labeled Protein A and L mixture in a 3605 plate. This unstressed FRET measurement was performed in duplicate. The ratio of labeled Protein A and L to antibody sample was optimized so that the unstress samples were at the top of the linear portion of the standard curve. The PCR plate was sealed with a Bio-Rad microseal B and place in a tetrad thermocycler to heat stress the plates for 10 minutes. After the temperature stress, the FRET signal of the stressed samples was measured in duplicate (20 μL of antibody mixture and 30 μL of the labeled Protein A and L mixture). The differences in stability was determined by comparing the ratios of the stressed and unstressed FRET signal for each sample. To compare mutations across different plates and backbones, the ratios was converted to a Performance Index (PI) by dividing the ratio of a variant by the ratio of the WT-backbone on the same plate.

Protease Stability: In a 3605 plate, 30 μL of concentrated T. reesei broth in 150 mM Na Acetate pH 5 was mixed with 70 μL of normalized antibody sample. The concentrated T. reesei broth was made by concentrating the broth using a 10 kDa MWCO membrane 10-fold. The T. reesei used to make the broth was an empty strain not expressing an antibody but did express background proteases. For the unstressed FRET read, 20 μL of this antibody mixture was added to 30 μL of the labeled Protein A and L mixture in a 3605 plate. This unstressed FRET measurement was performed in duplicate. The ratio of labeled Protein A and L to antibody sample was optimized so that the unstress samples were at the top of the linear portion of the standard curve. The stress plate was sealed with a Bio-Rad microseal B and place in an iEMS incubator at 30° C. for 2.5 hours. After the incubation, the FRET signal of the stressed samples was measured in duplicate (20 μL of antibody mixture and 30 μL of the labeled Protein A and L mixture). The differences in stability was determined by comparing the ratios of the stressed and unstressed FRET signal for each sample. To compare mutations across different plates and backbones, the ratios was converted to a Performance Index (PI) by dividing the ratio of a variant by the ratio of the WT-backbone on the same plate.

Results: Variants were tested for binding to Protein L, thermal stability, Tm, Fab stability and protease resistance. Heavy chain positions with PI improvement greater than 1 relative to the parent antibody sequence are shown in Table 1. Light chain positions with PI improvement greater than 1 relative to the parent antibody sequence are shown in Table 2.

TABLE 1
Heavy chain positions with PIs greater than 1 relative to parent antibody sequence

| | | Kabat | Constant | | | |
|---|---|---|---|---|---|---|
| Assay | Mutation* | position | domain† | Herceptin | VRC01 | HSV8 |
| Protease | 538N | 50 | | X | | |
| stability | | | | | | |
| Protease | 576S | 85 | | X | | |
| stability | | | | | | |
| Protease | 623Q | | 17 | X | | |
| stability | | | | | | |
| Protease | 708C | | 152 | X | | |
| stability | | | | | | |
| Protease | 712N | | 156 | X | | |
| stability | | | | | | |
| Protease | 713P | | 157 | X | | |
| stability | | | | | | |
| Protein L | 491I | 5 | | X | | X |
| Protein L | 575V | | 84 | X | | X |
| Protein L | 575Y | | 84 | X | | |
| Protein L | 708C | | 152 | X | | X |
| Thermal | 491T | 5 | | X | | X |
| stability | | | | | | |
| Thermal | 538N | 50 | | X | | |
| stability | | | | | | |
| Thermal | 624V | | 18 | | | X |
| stability | | | | | | |
| Thermal | 707L | | 151 | X | | X |
| stability | | | | | | |
| Thermal | 708E | | 152 | X | | X |
| stability | | | | | | |
| Fab | 491T | 5 | | X | X | X |
| Fab | 538N | 50 | | X | | X |
| Fab | 624V | | 18 | X | X | X |
| Fab | 707L | | 151 | X | X | X |
| Fab | 670V | | 64 | X | | X |
| Fab | 708D | | 152 | X | X | X |
| Fab | 708E | | 152 | X | X | X |
| Fab | 712N | | 156 | X | X | X |

*Position number corresponds to SEQ ID NO: 3
†Position number corresponds to SEQ ID NO: 1

TABLE 2
Light chain positions with PIs greater than 1 relative to parent antibody sequence

| | | Kabat | Constant | | | |
|---|---|---|---|---|---|---|
| Assay | Mutation* | position | domain† | Herceptin | VRC01 | HSV8 |
| Protein L | 527F | 42 | | X | | X |
| Protein L | 689M | | 97 | X | | X |
| Protein L | 689V | | 97 | X | | X |
| Fab | 673T | | 81 | X | | X |
| Fab | 692I | | 100 | | X | |

*Position number corresponds to SEQ ID NO: 4
†Position number corresponds to SEQ ID NO: 2

SEQUENCES

```
ASTKGPSVFP LAPSSKSTSG GTAALGCLVK DYFPEPVTVS WNSGALTSGV
                60 70 80 90 100
HTFPAVLQSS GLYSLSSVVT VPSSSLGTQT YICNVNHKPS NTKVDKKVEP
              110 120 130 140 150
KSCDKTHTCP PCPAPELLGG PSVFLFPPKP KDTLMISRTP EVTCVVVDVS
              160 170 180 190 200
HEDPEVKFNW YVDGVEVHNA KTKPREEQYN STYRVVSVLT VLHQDWLNGK
              210 220 230 240 250
EYKCKVSNKA LPAPIEKTIS KAKGQPREPQ VYTLPPSRDE LTKNQVSLTC
              260 270 280 290 300
LVKGFYPSDI AVEWESNGQP ENNYKTTPPV LDSDGSFFLY SKLTVDKSRW
              310 320 330
QQGNVFSCSV MHEALHNHYT QKSLSLSPGK (SEQ ID NO: 1)
RTVAAPSVFI FPPSDEQLKS GTASVVCLLN NFYPREARVQ WKVDNALQSG
                60 70 80 90 100
NSQESVTEQD SKDSTYSLSS TLTLSKADYE KHKVYACEVT HQGLSSPVTK
SFNRGEC (SEQ ID NO: 2)
QVTLRESGPALVKPTQTLTLTCTFSGFSLSTSGMSVGWIRQPPGKALEWLADIWWDD
KKDYNPS1KSRLTISKDTSKNQVVLKVTNMDPADTATYYCARSMITNWYFDVWGA
GTTVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDTRVEPKSCDKTH
TCPPCPAPEL1GGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK (SEQ
ID NO: 3)
DIQMTQSPSTLSASVGDRVTITCKCQLSVGYMHWYQQKPGKAPKLLIYDTSKLASG
VPSRFSGSGSGTEFTLTISSLQPDDFATYYCFQGSGYPFTFGGGTKLEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC (SEQ ID NO: 4)
Herceptin LC amino acid sequence
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEITRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC (SEQ ID NO: 5)
Herceptin HC amino acid sequence
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDTRVEPKSCDK
THTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
(SEQ ID NO: 6)
VRC01 LC amino acid sequence
EIVLTQSPGTLSLSPGETAIISCRTSQYGSLAWYQQRPGQAPRLVIYSGSTRAAGIPDR
FSGSRWGPDYNLTISNLESGDFGVYYCQQYEFFGQGTKVQVDIKRTVAAPSVFIFPPS
DEQLKSGTASVVCLLNNFYPREAKVQWKVDNALQSGNSQESVTEQDSKDSTYSLSS
TLTLSKADYEKHKVYACEVTHQGLSSPVTKSFNRGEC (SEQ ID NO: 7)
VRC01 HC amino acid sequence
QVQLVQSGGQMKKPGESMRISCRASGYEFIDCTLNWIRLAPGKRPEWMGWLKPRG
GAVNYARPLQGRVTMTRDVYSDTAFLELRSLTVDDTAVYFCTRGKNCDYNWDFEH
WGRGTPVIVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPG
(SEQ ID NO: 8)
```

We claim:

1. An isolated monoclonal antibody or functional fragment thereof comprising:
(a) a heavy chain variable region comprising one or more amino acid substitution(s) comprising T or an I at Kabat position 5; N at Kabat position 50, V or Y at Kabat position 84; and/or S at Kabat position 85; and or
(b) a heavy chain constant region comprising one or more amino acid substitution(s) comprising Q at position 17; V at position 18; V at position 64; L at position 151; C, E, or D at position 152; N at position 156; and/or P at position 157, wherein the amino acid positions of the heavy chain constant region correspond to those of SEQ ID NO:1, and wherein said antibody exhibits one or more improved properties comprising increased manufacturability, thermostability, and/or protease resistance compared to an antibody that does not comprise the one or more amino acid substitutions.

2. The antibody or functional fragment thereof of claim 1, further comprising:
(c) a light chain variable region comprising an amino acid substitution comprising F at Kabat position 42; and/or
(d) a light chain constant region comprising one or more amino acid substitution(s) comprising T at position 81; M or V at position 97; and/or I at position 100, wherein the amino acid positions of the light chain constant region correspond to those of SEQ ID NO:2.

3. An isolated monoclonal antibody or functional fragment thereof comprising:
(c) a light chain variable region comprising an amino acid substitution comprising F at Kabat position 42; and/or
(d) a light chain constant region comprising one or more amino acid substitution(s) comprising T at position 81; M or V at position 97; and/or I at position 100, wherein the amino acid positions of the light chain constant region correspond to those of SEQ ID NO:2, wherein said antibody exhibits one or more improved properties comprising increased manufacturability, thermostability, and/or protease resistance compared to an antibody that does not comprise the one or more amino acid substitutions.

4. The antibody or functional fragment thereof of claim 3, further comprising:
(a) a heavy chain variable region comprising one or more amino acid substitution(s) comprising T or an I at Kabat position 5; N at Kabat position 50, V or Y at Kabat position 84;
and/or S at Kabat position 85; and or
(b) a heavy chain constant region comprising one or more amino acid substitution(s) comprising Q at position 17; V at position 18; V at position 64; L at position 151; C, E, or D at position 152; N at position 156; and/or P at position 157, wherein the amino acid positions of the heavy chain constant region correspond to those of SEQ ID NO:1.

5. The antibody or functional fragment thereof of any one of claims 1-4, wherein said functional fragment is selected from the group consisting of Fab, Fab′, F(ab′)2 and Fv fragments.

6. The antibody or functional fragment thereof of any one of claims 1-4, wherein said antibody is chimeric, humanized, or fully human.

7. The antibody or functional fragment thereof of any one of claims 1-6, wherein said antibody is selected from the group consisting of trastuzumab, an anti-HSV8 antibody, and VRC01.

8. The antibody or functional fragment thereof of any one of claims 1-6, wherein said antibody competitively inhibits the binding of one or more of trastuzumab, an anti-HSV8 antibody, and VRC01 to an antigen.

9. A nucleic acid encoding the heavy chain variable region and/or heavy chain constant region of claim 1.

10. A nucleic acid encoding the light chain variable region and/or light chain constant region of claim 3.

11. A vector comprising the nucleic acid of claim 9 and/or claim 10.

12. A recombinant cell comprising the vector of claim 11.

13. The recombinant cell of claim 12, wherein the cell is a mammalian cell, a bacterial cell, or a fungal cell.

14. The recombinant cell of claim 13, wherein the fungal cell is T. reesei.

15. A method for improving one or more properties in a monoclonal antibody comprising:
(a) introducing one or more substitutions comprising T or an I at Kabat position 5; N at Kabat position 50, V or Y at Kabat position 84; and/or S at Kabat position 85 in a heavy chain variable region; and/or
(b) introducing one or more substitutions comprising Q at position 17; V at position 18; V at position 64; L at position 151; C, E, or D at position 152; N at position 156; and/or P at position 157, in a heavy chain constant region wherein the amino acid positions of the heavy chain constant region correspond to those of SEQ ID NO:1.

16. The method of claim 15, further comprising:
(c) introducing one or more substitutions comprising F at Kabat position 42 in a light chain variable region; and/or
(d) introducing one or more substitutions T at position 81; M or V at position 97; and/or I at position 100 in a light chain constant region, wherein the amino acid positions of the light chain constant region correspond to those of SEQ ID NO:2.

17. A method for improving one or more properties in a monoclonal antibody comprising:
(c) introducing a substitution comprising F at Kabat position 42 in a light chain variable region; and/or
(d) introducing one or more substitution(s) comprising T at position 81; M or V at position 97; and/or I at position 100 in a light chain constant region, wherein the amino acid positions of the light chain constant region correspond to those of SEQ ID NO:2.

18. The method of claim 17, further comprising:
(a) introducing one or more substitutions comprising T or an I at Kabat position 5; N at Kabat position 50, V or Y at Kabat position 84; and/or S at Kabat position 85 in a heavy chain variable region; and/or
(b) introducing one or more substitutions comprising Q at position 17; V at position 18; V at position 64; L at position 151; C, E, or D at position 152; N at position 156; and/or P at position 157, in a heavy chain constant region wherein the amino acid positions of the heavy chain constant region correspond to those of SEQ ID NO:1.

19. The method of any one of claims 15-18, wherein said improved properties is one or more of increased manufacturability, thermostability, and/or protease resistance.

20. The method of any one of claims 15-19, wherein said antibody is selected from the group consisting of trastuzumab, an anti-HSV8 antibody, and VRC01.

21. A method for producing a monoclonal antibody or functional fragment thereof comprising providing an isolated cell with a nucleic acid encoding said antibody or functional part thereof, wherein said antibody or functional part thereof comprises one or more of:
(a) a heavy chain variable region comprising one or more amino acid substitution(s) comprising T or an I at Kabat position 5; N at Kabat position 50, V or Y at Kabat position 84; and/or S at Kabat position 85;
(b) a heavy chain constant region comprising one or more amino acid substitution(s) comprising Q at position 17; V at position 18; V at position 64; L at position 151; C, E, or D at position 152; N at position 156; and/or P at position 157, wherein the amino acid positions of the heavy chain constant region correspond to those of SEQ ID NO:1
(c) a light chain variable region comprising an amino acid substitution comprising F at Kabat position 42; and/or
(d) a light chain constant region comprising one or more amino acid substitution(s) comprising T at position 81; M or V at position 97; and/or I at position 100, wherein the amino acid positions of the light chain constant region correspond to those of SEQ ID NO:2.

22. The method of claim 21, wherein the cell is a mammalian cell, a bacterial cell, or a fungal cell.

23. The method of claim 22, wherein the fungal cell is T. reesei.

24. The method of any one of claims 21-23, wherein the antibody or functional fragment thereof exhibits one or more improved properties selected from the group consisting of increased manufacturability, thermostability, and protease resistance compared to an antibody that does not comprise one or more of the amino acid substitutions.

25. The method of any one of claims 21-24, wherein said antibody is selected from the group consisting of trastuzumab, an anti-HSV8 antibody, and VRC01.

26. A pharmaceutical composition comprising the antibody or functional fragment thereof of any one of claims 1-8 and a pharmaceutically acceptable carrier, diluent, or excipient.
